(19) United States
(12) Patent Application Publication
(10) Pub. No.: US 2018/0281024 A1
(43) Pub. Date: Oct. 4, 2018

(54) SYSTEM AND METHOD FOR PIERCING CONTAINERS

(71) Applicant: AMT Group Bahamas Ltd., Lyford Cay (BS)

(72) Inventor: Gregory John Dobbyn, Creedmoor, NC (US)

(21) Appl. No.: 15/939,817

(22) Filed: Mar. 29, 2018

Related U.S. Application Data
(60) Provisional application No. 62/478,374, filed on Mar. 29, 2017.

(51) Int. Cl. B08B 3/04; B67B 7/00; A61L 2/16

(52) U.S. Cl. CPC B08B 3/04; A61L 2202/24; A61L 2/16; B67B 7/24

(57) ABSTRACT

Piercing one or more containers (e.g., single use containers) within a cleaning and disinfecting device is provided. An actuator (e.g., motor, or the like) causes drive mechanisms to move a member (e.g., tube) and/or a piercer linearly with respect to a longitudinal axis of the member. The actuator moves the member towards a container until the piercer has pierced a portion of the container. A guide may form a seal with a reservoir located under the container to create a seal with the reservoir and keep contents of the container from passing from a reservoir to components of a piercer apparatus. Alternatively, the piercer and/or guide form a seal with the container, and the contents pass from the container to an outlet channel in the member, such that the contents of the container are emptied out of the container to the outlet channel and directly or indirectly to a medical device.

### CROSS REFERENCE AND PRIORITY CLAIM UNDER 35 U.S.C. § 119

The present Application for a Patent claims priority to U.S. Provisional Patent Application Ser. No. 62/478,374 entitled “System and Method for Piercing Containers” filed on Mar. 29, 2017 and assigned to the assignees hereof and hereby expressly incorporated by reference herein.

### FIELD

This application relates generally to the field of automated piercing devices, and more particularly to automated piercing devices for cleaning and/or disinfecting medical devices, specifically for piercing containers for cleaning and disinfecting probes.

### BACKGROUND

Some medical devices, such as probes, and in particular Transesophageal Echocardiograph (TEE) probes, which are channel-less ultrasound transducer probes that are inserted down the esophagus of a patient, cannot undergo sterilization (e.g., because of the electronic components) and do not contact sterile areas of the human body. Since these probes and other medical devices cannot be sterilized using traditional methods, high-level disinfection of these types of probes is required. Some such high-level disinfection types require a step of using a cleaning or disinfectant container, which may be utilized during specific steps of the cleaning and disinfecting process.

### BRIEF SUMMARY

Embodiments of the invention comprise a piercing apparatus having a piercer assembly and a drive assembly that is used to engage and disengage a piercer from a container. In some embodiments of the invention, the drive assembly is operatively coupled to the piercer assembly (e.g., directly to a piercer, or to a piercer member coupled to the piercer in the piercer assembly) to move the piercer into one or more engaging and disengaging positions. The drive assembly may include an actuator, such as a motor, that is operatively coupled to drive mechanisms that adjust the piercer and/or piercer member (e.g., one or more piercer members) into and out of the engaging and disengaging positions. In some aspects of the invention the drive mechanisms may include a piston, a rack and pinion, a gear assembly, a lever configuration, a belt and screw configuration (as will be described in further detail with respect to FIGS. 1-5), a pneumatic and/or hydraulic system, a friction drive, and/or other like drive mechanisms that move the piercer directly, a piercer member operatively coupled to the piercer, or the like. The piercer may pierce a container and allow the contents of the piercer to flow into a reservoir for delivery to a cleaning and/or disinfecting device. In some embodiments, of the invention the piercer assembly may include the piercer and a piercer member that is a tube. The tube may be hollow, and as such the contents of the container may flow through the tube and be delivered to the desired location, such as for cleaning and/or disinfecting of medical devices. As such, the member (e.g., the tube) may be a hollow or solid member of any shape, such as circular, rectangular, square, oval, or the like.

Embodiments of the invention may be directed specifically to apparatuses and methods for piercing containers used in cleaning and disinfecting apparatuses for medical devices, particularly for cleaning and disinfecting probes (e.g., TEE Probes, or the like). As such, an actuator, such as a motor or other actuator mechanism may cause a lead screw to rotate in a first direction, thereby causing a guided nut to travel linearly along the longitudinal axis of the lead screw. The guided nut may be operatively coupled (e.g., clasped, glued, welded, or the like) to a tube that travels along substantially the same path as the guided nut. The top portion of the tube may comprise a guide, one or more sealing members (e.g., gaskets and/or O-rings), and a piercer (e.g., a sharp object) capable of piercing (e.g., cutting, shearing, punching, or otherwise opening) a container.

As the motor operates in a first configuration, the tube, along with the piercer, may be moved upward such that the piercer pierces the container or a container cover of the container. One or more position sensors may monitor the linear movement of the piercing apparatus (e.g., location of the piercer, location of the member, actuation of the actuator, or the like), and will determine that the piercing apparatus is in an engaged position. In some embodiments, a position sensor transmits one or more control signals configured to cause the motor to stop, cause the motor to move, or otherwise locks the motor to lock piercing apparatus in place.

In other embodiments, in the engaged position at least a portion of the piercer is positioned at least partially within the container (i.e., after piercing the container). Typically a sealing member (e.g., a reservoir sealing member) is used to seal the piercing assembly and the reservoir, such as sealing the piercing assembly with the reservoir between a reservoir opening and a guide of the piercing assembly. In other aspects of the invention, such as when the piercer assembly allows for the flow of the contents through the member (e.g., tube), a container seal associated with the piercer may seal the container to create an air-tight and/or fluid-tight seal with the container. In this configuration, the tube comprises an outlet channel defined by the walls of the tube that is configured to accept the contents of the container as the contents empty due to the piercing of the container. The outlet channel of the tube may direct the contents of the container to a specific reservoir, or may transfer the contents of the container to a hose or other channel configured to direct the contents of the tube to the appropriate reservoir.

In some embodiments, the piercing apparatus remains in the engaged position for a predetermined period of time (e.g., for as long as it is expected for the contents of the container to drain out), before the motor begins operating in a second configuration to rotate the lead screw in the opposite direction and cause the member (e.g., tube, or the like) and piercer to retreat from the container and return to a normal or resting position.

In other embodiments, one or more content sensors (e.g., wet/dry sensors) may determine whether the contents of the container are still present in a certain portion of the apparatus. When the content sensors determine that the contents of the container are no longer present (which indicates that the container has been emptied), the content sensors may automatically transmit control signals configured to cause the motor to operate in its second configuration and return the piercer and tube back to normal or resting positions.

The piercing apparatus and methods of use can also be used in conjunction with apparatuses and methods for cleaning and disinfecting specific medical devices, such as TEE probes. In some such embodiments, a cleaner is first utilized to remove foreign material (e.g., bioburden, soil, and the like) from the probe after the probe is removed from the patient by soaking and/or flushing the probe with the cleaner. The cleaner may be a detergent or a detergent with one or more enzymes to enhance cleaning. The multiple enzymes in the cleaner rapidly attack soils, and include low foam properties for effective recirculation for various cycles of cleaning the probe. The cleaner may or may not be applied through the use of a container and/or a piercing apparatus. The probe is rinsed after the cleaning step to remove the residual cleaner from the probe and from within the fluid circuit. After rinsing, a high-level disinfection process is applied to the probe. The high-level disinfectant soaks and/or flushes the probe for a specified amount of time, and afterward, the probe is thoroughly rinsed again to remove the disinfectant from the probe and from within the fluid circuit. The disinfectant may or may not be applied through the use of a container (e.g., single use container, or the like) and/or a piercing apparatus.

Embodiments of the invention comprise an apparatus for piercing a container. The apparatus comprises an actuator and a drive mechanism operatively coupled to the actuator. Moreover, a piercer assembly is operatively coupled to the drive mechanism, wherein the piercer assembly comprises at least a piercer, and wherein the piercer is configured to pierce the container, thereby causing contents of the container to exit the container.

In further accord with embodiments of the invention, a first operational configuration of the actuator directs the drive mechanism to move the piercer towards the container.

In other embodiments the invention further comprises one or more position sensors configured to determine when the piercer is in the engaged position by measuring a position of the piercer or by measuring an amount of actuation of the actuator.

In yet other embodiments of the invention, when the piercer is in an engaged position with the container at least a portion of the piercer has pierced the container and is located within the container.

In still other embodiments the piercer assembly further comprises a guide and a reservoir seal, and wherein the guide and the reservoir seal are operatively coupled a reservoir associated with the container and seal the piercer assembly with respect to the reservoir.

In further accord with embodiments of the invention, the piercer assembly further comprises a piercer member operatively coupled to the piercer, and wherein the piercer member comprises a tube and an outlet channel within the tube configured to receive contents from the container when the piercer is in the engaged position.

Embodiments of the invention further comprise a method for piercing a container. The method comprises moving a piercing assembly, wherein the piercing assembly is moved toward the container, and the piercing assembly comprises at least a piercer. The method further comprises determining that the piercer is in an engaged position, wherein the engaged position occurs when the piercer has pierced a portion of the container. Thereafter the method comprises providing contents of the container that exit the container to a medical device, and retracting the piercer from the engaged position to a disengaged position.

In further accord with embodiments of the invention, moving the piercing assembly further comprises manually moving the piercing assembly toward the container.

In other embodiments of the invention, moving the piercing assembly further comprises moving the piercing assembly through a drive assembly, wherein the drive assembly comprises an actuator operatively coupled to a drive mechanism, and wherein the piercer is operatively coupled to the drive mechanism.

In yet other embodiments, the piercing assembly further comprises a guide and a reservoir seal, and the reservoir seal and the guide seal a portion of the piercing assembly within the reservoir. Moreover, providing the contents of the container to the medical device comprises allowing the contents to flow into the reservoir associated with the container. The reservoir is configured to collect the contents of the container and direct the contents of the container to the medical device.

In still other embodiments, the piercing assembly further comprises a piercer member operatively coupled to the piercer, a guide, a container seal, and an outlet channel in the piercing member. Moreover, providing the contents of the container to the medical device comprises allowing the contents to flow into the outlet channel of the piercer member, and wherein the outlet channel is configured to receive the contents of the container directly from the container and direct the contents of the container to the medial device.

Embodiments of the invention comprise an apparatus for cleaning and disinfecting a probe, the apparatus comprises a probe reservoir assembly, wherein the probe reservoir assembly is configured for securing a probe for the cleaning and the disinfecting. The apparatus further comprises a cleaner assembly operatively coupled to the probe reservoir assembly, wherein the cleaner assembly is configured for providing a cleaner to the probe reservoir assembly for the cleaning of the probe before the disinfecting. The apparatus further comprises a disinfectant assembly operatively coupled to the probe reservoir assembly, wherein the disinfectant assembly comprises a piercing apparatus comprising a piercing assembly, and wherein the piercing assembly is configured for piercing a container of a disinfectant and providing the disinfectant for the disinfecting of the probe after the cleaning.

In further accord with embodiments of the invention, the piercing assembly is configured for manual movement.

In other embodiments, the piercing apparatus further comprises a drive assembly configured for automatically moving the piercing assembly for piercing the container.

In yet other embodiments, the drive assembly comprises an actuator and a drive mechanism operatively coupled to the actuator. Moreover, the piercer assembly comprises a piercer member operatively coupled to the drive mechanism, a piercer operatively coupled to the member, and wherein the piercer is configured to pierce the container to allow contents of the container exit the container.

In still other embodiments, a first operational configuration of the actuator directs the drive mechanism to move the piercer towards the container.

In further accord embodiments of the invention further comprise one or more position sensors configured to measure a position of the member or the piercer, or an amount of actuation of the actuator.

In other embodiments of the invention, the piercer assembly further comprises a guide and a reservoir seal, and wherein the guide and the reservoir seal are operatively coupled to the probe reservoir assembly and seal a portion of the piercer assembly with respect to the probe reservoir assembly.

In still other embodiments of the invention, the guide comprises a male fitting and a female fitting and is operatively coupled to the member to allow the member to slide within the guide.

In yet other embodiments of the invention, the piercer member is a tube and further comprises an outlet channel within the tube configured to receive contents from the container when the piercer is in the engaged position.

To the accomplishment the foregoing and the related ends, the one or more embodiments comprise the features hereinafter described and particularly pointed out in the claims. The following description and the annexed drawings set forth certain illustrative features of the one or more embodiments. These features are indicative, however, of but a few of the various ways in which the principles of various embodiments may be employed, and this description is intended to include all such embodiments and their equivalents.

### BRIEF DESCRIPTION OF THE DRAWINGS

Having thus described embodiments of the invention in general terms, reference will now be made to the accompanying drawings, where:

FIG. 1 illustrates a high level process flow for piercing a container with an automated piercer device, in accordance with embodiments of the invention;

FIG. 2 illustrates a perspective view of the automated piercer device, in accordance with embodiments of the invention;

FIG. 3 illustrates a side view of the automated piercer device, in accordance with embodiments of the invention;

FIG. 4 illustrates a top view of the automated piercer device, in accordance with embodiments of the invention;

FIG. 5 illustrates a side view of a cleaning and disinfecting apparatus comprising the automated piercer device of FIG. 2, in accordance with embodiments of the invention; piercer

FIG. 6 illustrates a high level process flow for the automatic cleaning and disinfecting of probes, in accordance with embodiments of the invention;

FIG. 7 illustrates a front view of the cleaning and disinfecting apparatus, in accordance with embodiments of the invention;

FIG. 8 illustrates a top view of the cleaning and disinfecting apparatus, in accordance with one embodiment of the invention;

FIG. 9 illustrates a rear view of the cleaning and disinfecting apparatus with the housing removed, in accordance with embodiments of the invention;

FIG. 10 illustrates a side view of the cleaning and disinfecting apparatus, in accordance with embodiments of the invention; and

FIG. 11 illustrates a schematic diagram of the fluid flow within the cleaning and disinfecting apparatus, in accordance with embodiments of the invention.

### DETAILED DESCRIPTION OF EMBODIMENTS OF THE INVENTION

Embodiments of the present invention now may be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure may satisfy applicable legal requirements. Like numbers refer to like elements throughout.

The apparatuses and methods of the present invention are described generally as providing an automated piercing apparatus and methods of operating the piercing apparatus to pierce a container, either separately as a stand-alone containers, or in conjunction with a larger apparatus. For example, the piercing apparatus and methods described herein may be used in specific applications, such as for piercing cleaning and/or disinfectant containers to clean medical devices within a cleaning and/or disinfecting apparatus. In some embodiments, the piercing apparatus and methods of using the piercing apparatus may be used in conjunction with cleaning and/or disinfecting one or more TEE probes.

Particularly, in one embodiment the present invention is directed to apparatuses and methods of use that allows for the assembly of a container with a cleaning and/or disinfecting apparatus for use in cleaning and/or disinfecting a medical device before the cleaning and/or disinfecting is to occur. That is, the container may be assembled for use at a future point in time.

Moreover, the present invention is directed to apparatuses and methods for piercing a container in such a way that the piercing apparatus and the sides of the container prevent or minimize splashing, leaking, and general spilling of liquid, solid, and/or gas contents of the container, and for preventing the same from passing outside of the reservoir or through to any portion of the piercing apparatus (e.g., except for through one or more outlet channels defined by the piercing assembly in some embodiments of the invention). In some embodiments of the invention, the piercer of the automated piercing apparatus opens the container and allows the contents of the container to flow into a reservoir for delivery to a medical device for cleaning and/or disinfecting. In some embodiments of the invention, a seal between the automated piercing apparatus and the reservoir is formed to prevent contents of the container (e.g., disinfectant material, cleaning material, rinsing fluid, and the like) from interacting with other portions of a general cleaning and disinfecting apparatus. The piercing apparatus limits the cleaning and/or disinfecting apparatus' exposure to potent chemicals and other cleaning and/or disinfecting solutions that should remain medically sterile, or at least should remain free of interaction with the contents outside of the container.

Additionally, an outlet channel of the reservoir (or an outlet channel within a member in the piercing apparatus), when used in combination with the sealing features of the piercing apparatus, ensures that substantially all of the contents of the container pass on to one or more other portions of a cleaning and/or disinfecting apparatus for cleaning and/or disinfecting a medical device. As such, operators of the piercing apparatus can be sure that a known or expected amount of the contents of the container have entered a cleaning and/or disinfecting portion of the cleaning and/or disinfecting apparatus. This knowledge allows a medical professional to be confident that a medical device, such as a probe, being cleaned or sterilized in the cleaning and disinfecting apparatus has received an appropriate amount of disinfectant and/or cleaning solution from the container, without requiring additional measurements.

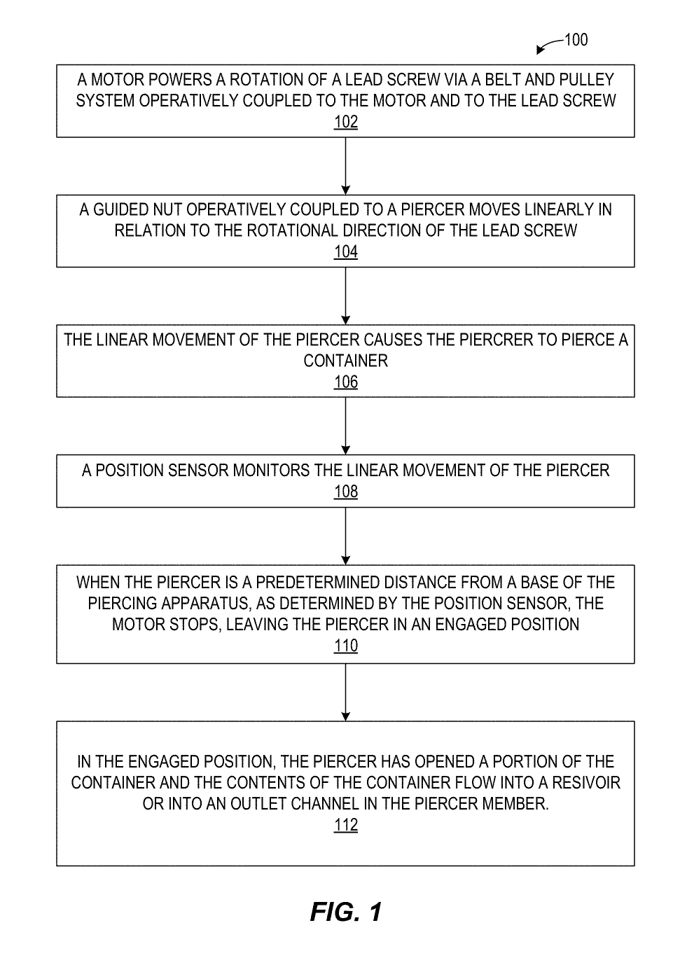

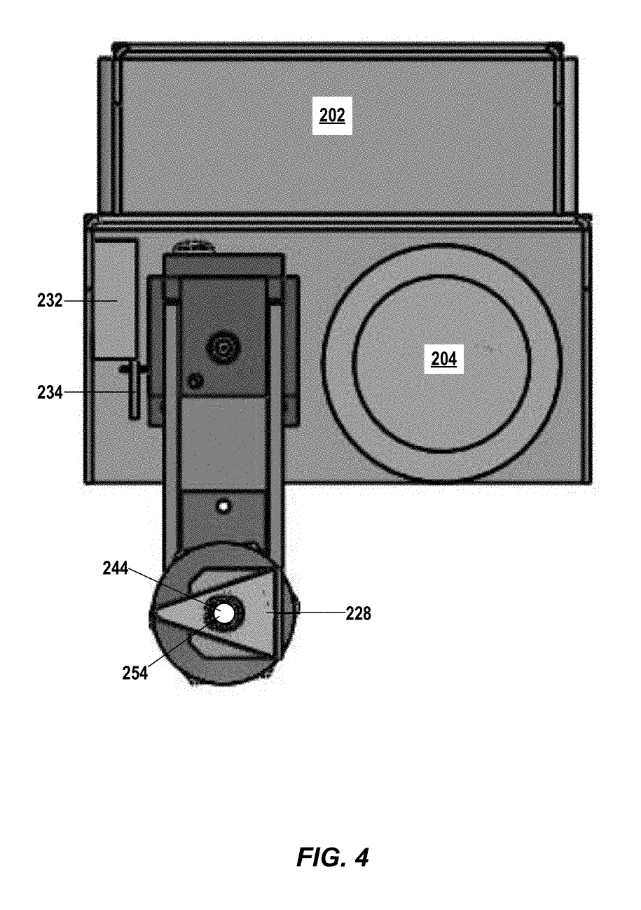

Generally, the piercing apparatus comprises a drive assembly that is used to engage and disengage a piercer from a container. In some embodiments of the invention, the drive assembly is operatively coupled to the piercer (or member coupled to the piercer) to move the piercer into, or out of, one or more engaging and disengaging positions. The drive assembly may include an actuator (e.g., a motor, or the like) that is operatively coupled to drive mechanisms and a member (e.g., one or more members) that moves the piercer into and out of the engaging and disengaging positions. In some aspects of the invention, the drive mechanisms may include a piston, a rack and pinion, a gear assembly, a lever configuration, a belt and screw configuration (as will be described in further detail with respect to FIGS. 1-5), a pneumatic and/or hydraulic system, a friction drive, and/or other like drive mechanisms that move the piercer directly, or a member operatively coupled to the piercer. In some embodiments of the invention, the member may include a tube. The tube may be a hollow or solid member of any shape, such as circular, rectangular, square, oval, or the like.

In some aspects of the invention the drive assembly may be configured such that the drive mechanism causes the piercer, directly or through the use of one or more members, to move substantially linearly along a longitudinal axis of the piercer and/or one or more members. In some embodiments, the drive assembly is configured to cause the piercer and/or the one or more members to move in a rotated direction, spiral direction, diagonal direction, vertical direction, horizontal direction, and the like to pierce a container.

When the drive assembly is in a first operating state, the drive mechanism causes the piercer, directly or through a member, to move in a direction toward a container. The piercer may have an edge that can create an aperture in the container (e.g., sharp, pointed, or the like edge). As such, the piercer may be a tang, a knife, a wedge, a screw, a nail, or other like component that is operatively coupled to an end of the member, or operatively coupled directly to the drive mechanism. Therefore, when the drive assembly, operating in the first operating state, drives the piercer, directly or through a member, toward the container, the piercer creates an aperture in the container, such as through a container cover. The piercer may be used to pierce (e.g., separate, split, shear, puncture, slice, or otherwise open) a portion of the container such that contents of the container may empty out of the container. In some embodiments, the piercer may pierce a bottom of the container (as it is positioned within an apparatus), such that the contents of the container are emptied out of the container due to gravity.

A position sensor may cause the drive assembly to stop moving the piercer (or the member) once the position sensor determines that the piercer (or the member), are in an engaged position with the container. The engaged position may comprise a position where the piercer has pierced the container, one or more sealing members (e.g., a gasket, an O-ring, and the like) have formed seals between the piercer and the container, seals between the guide and a reservoir associated with the container, and/or between the member and the guide (e.g., a container seal, a reservoir seal, or a tube seal). The drive assembly may then operate in a second operating state (before or after all the contents from the container have drained) configured to cause the member and/or piercer to retract from the engaged position and return to a disengaged or resting position. The used container may be removed, and a new, unpierced, container may replace the used container for a future cycle of the piercing assembly.

The piercing cycle can be used to automatically pierce containers in many different systems. In some embodiments, the piercing apparatus is used to pierce a cleaning and/or disinfecting container within a medical device cleaning and disinfecting apparatus. The automatic piercing of the cleaning and/or disinfecting container allows cleaning and/or disinfecting contents of the container to empty from the container and flow into a reservoir for delivery to a medical device, and/or flow through tubing to interact with the medical device (e.g., a probe, surgery equipment, implant material, and the like).

The cleaning and/or disinfecting container may be loaded into a container receptor of the medical device cleaning and/or disinfecting apparatus, such that the container is not pierced until the piercing system is turned on. For example, the medical device cleaning and/or disinfecting apparatus may be a mobile apparatus that can be carried or otherwise moved by a user. A medical device (e.g., a probe, or other device) may be inserted into a medical device housing portion of the apparatus, and the cleaning and/or disinfecting container may be loaded into the apparatus. In some embodiments, the cleaning and/or disinfecting container may be loaded into the cleaning and/or disinfecting apparatus ahead of time, in anticipation of a future cleaning and disinfecting of a medical device. The container may remain closed until the medical device is inserted and ready to be cleaned and/or disinfected. In this way, the contents of the container remain fresh, are not unintentionally diluted, do not contaminate other portions of the apparatus, and are not contaminated by portions of the apparatus before the contents are desired to be introduced to the medical device.

In some embodiments, a user may turn the cleaning and/or disinfecting apparatus on, and the apparatus automatically causes the piercing apparatus to open the container at a desired time after turning on the apparatus. In other embodiments, a user may manually determine when the container should be pierced and operates controls on the cleaning and/or disinfecting apparatus to turn on the piercing apparatus. The piercing apparatus may then automatically operate in its first operating state to cause the piercer, directly or through the member, to move toward the container, to pierce the container (e.g., a container foil, or the like), and the position sensors determine when to stop the movement of the piercer, directly or through the member) based when they are in the engaged position.

In some embodiments, the piercing apparatus may be manually controlled to pierce the container. For example, a user may rotate a lever operatively coupled to at least a portion of the piercing apparatus substantially towards (e.g., upward) the container with enough force to cause the piercer portion of the piercing apparatus to pierce through the container cover (or another portion of the container). In some embodiments, the user may use a handle operatively coupled to a portion of the piercing apparatus (e.g., a handle extending from the tube of the piercing apparatus and extending outside of the disinfecting apparatus) to slide or otherwise reposition the piercing apparatus within the disinfecting apparatus until the piercing apparatus engages with or otherwise pierces the container. In some embodiments, a button, rope, or the like may be operatively coupled to a portion of the piercing apparatus and/or may extend outside of the disinfecting apparatus, such that a user may pull or move the piercing apparatus with respect to the container in order to pierce the container. In some embodiments, the user may push (e.g., upward, downward, laterally, and/or the like) on a slide operatively coupled to the piercing apparatus, causing the piercing apparatus to move upward and pierce the container cover.

The piercing apparatus is generally described herein as being the moveable feature that allows for the piercing of the container. However, it should be understood that in some embodiments, the container may be inserted into the cleaning and/or disinfecting apparatus to an installed position. The piercing apparatus may be stationary, and as such, the container may be moved (e.g., pushing on the container) into a piercing position which allows the piercing apparatus to pierce the container. For example, the container may be located in the installed position within the container, a safety may be released (e.g., a lock that allows the container to move, a cover may be removed or repositioned to expose the piercing apparatus, enough force is applied to the container to deform the container to allow it to contact the piercing apparatus, or the like). It should be understood that the container may be moved (e.g., automatically or manually) utilizing one or more of the means described herein with respect to moving the piercer. As such, the container may be moved into a piercing position from the installed position, such that the container may be pierced in order to begin the cleaning and/or disinfection process.

It should be understood that the container may be a single container that is used for disinfecting and/or cleaning; there may be multiple containers, which may include one container for disinfecting and one container for cleaning; or there may be a single container that is used only for disinfecting while the cleaning is performed through another mechanism as described below.

In some embodiments, the piercing apparatus is used in a TEE probe cleaning and disinfecting apparatus, where the piercing apparatus automatically pierces a disinfecting container to release disinfectant within the apparatus to disinfect and otherwise sterilize a TEE probe after it has been used in a medical operation.

FIG. 1 illustrates a high level process flow 100 for piercing a container with a specific type of piercing apparatus 200 described herein. As illustrated by block 102, a motor powers a rotation of a lead screw via a belt and pulley system operatively coupled to the motor and to the lead screw. The motor, when operating in a first configuration, causes the belt to rotate the lead screw in a first direction. When the motor is operated in a second configuration, causes the belt to rotate the lead screw in a second direction (e.g., an opposite direction to the first direction). The motor may also operate in a third configuration where the motor is either turned off, does not cause movement of the belt, or locks the belt (and therefore the lead screw) in place.

As illustrated by block 104 of FIG. 1, a guided nut that is operatively coupled to a piercer moves linearly in relation to the rotational direction of the lead screw. The guided nut may be operatively coupled to the lead screw, such that the interior ridges of the guided nut align with the angled ridges of the lead screw. The guided nut may also abut against a wall, guide, or other siding of a carriage that houses the lead screw, such that the guided nut does not rotate with the lead screw, but slides along the wall of the carriage as the ridges (e.g., threads) of the lead screw push the guided nut. Therefore, as the lead screw is rotated by the motor, the nut travels in a linear path along the lead screw. As the guided nut is also operatively coupled to the piercer, directly or through the use of a piercing member (e.g., tube), the piercer also travels longitudinally along substantially the same path as the guided nut, in response to the directional rotation of the lead screw as controlled by the motor.

In some embodiments, the linear movement of the piercer causes an edge of the piercer to pierce a container (e.g., foil of a container), as shown in block 106 of FIG. 1. As the piercer travels upward, it approaches, contacts, and eventually breaches a container. Therefore, the piercer may comprise an edge with sharp portion capable of piercing a container. In some embodiments, the piercer pierces a side of the container, while in other embodiments, the container comprises a container opening cover (e.g., a foil seal) that can be easily pierced by the piercer. By piercing the container, the piercer causes contents of the container to flow or otherwise exit the container.

In some embodiments, the motor may be another type of actuator, and as such the actuator may be hydraulic, pneumatic, manual, or be another like means for causing a belt, or other like feature, to rotate the lead screw, thereby moving the piercer. In other embodiments, the means for moving the piercer described in blocks 102 to 106 may not utilize a screw, guide nut, or the like. Instead, the actuator (e.g., motor, hydraulic, pneumatic, manual, or another like actuator) may utilize other features, such as a piston rod, linkages, or the like the pierce the container.

Furthermore, in some embodiments of the process 100, a position sensor monitors the linear movement of the piercer, as shown in block 108 of FIG. 1. The position sensor can monitor the location of one or more components of the piercer, speed, acceleration, or other like information associated with one or more components of the piercer. The position sensor can also control the motor (either directly or through a communication network) based on the measurements taken by the sensor.

When the position sensor indicates that the piercer is a predetermined distance from a base of the piercing apparatus (or at a location based on another measurement), the motor stops, which leaves the piercer in an engaged position, as described in block 110 of FIG. 1. Once the position sensor determines that the piercer is in its engaged position, the position sensor can transmit a signal configured to cause the motor to shut down or otherwise lock the lead screw in place, such that the piercer is also locked in its engaged position. In other embodiments, the one or more sensors may be operatively coupled to the actuator, or otherwise positioned to take measurements associated with the actuation of the actuator. For example, one or more actuator sensors may be configured to determine and measure the rotation or movement of the actuation of the actuator, which may be directly correlated to the length of the piercer and/or piercer member. In this way, the actuator sensors are able to determine a position of the piercer based on actuation or rotation measurements of the actuator.

As illustrated by block 112 of FIG. 1, as the piercer is moved into the engaged position, and pierces the container (e.g., a container surface, container cover, or the like), the contents of the container flow out the container and into a reservoir for ultimate delivery to a medical device (as will be explained in further detail later with respect to the TEE probe cleaning and/or disinfecting apparatus). One or more sealing members are used to seal the connection between reservoir and piercer apparatus. For example, a reservoir seal seals the interface between the guide and the reservoir, while a tube seal seals the interface between the guide and the tube that allows the tube to slide within the guide. In this configuration, the sealing members of the piercer assembly may be pressed between the wall of the reservoir and the guide, and between the guide and the tube, in order to form an air-tight and/or fluid-tight seal between the components such that the contents of the container do not spill out of the reservoir and into other components of the piercer apparatus (e.g., the motor, the lead screw, and the like) or other parts of the cleaning and/or disinfecting apparatus.

In other embodiments of the invention, as the piercer is moved into the engaged position, another sealing member (e.g., gasket, o-ring, or the like) forms a seal between the piercer assembly and the container (e.g., a container seal). As such, the sealing member forms a container seal with the container and provides an outlet channel defined within the piercer and/or piercer member to transport the contents of the container to a portion of the cleaning and/or disinfecting apparatus that should receive the contents of the container. To accomplish the sealing aspects of this embodiment, the sealing member operatively coupled to the piercer may form an air-tight and/or fluid-tight seal between the piercer and a portion of the container when in the engaged position, such that contents of the container cannot pass through any gaps between the two. The piercer may comprise an aperture and a member operatively coupled to the piercer. The member may have an outlet channel running throughout at least a portion of the member that is capable of receiving the contents of the container once the container is pierced at a first location (e.g., first end). Therefore, substantially all of the contents of the container are emptied out through the outlet channel of the member coupled to the piercer when the piercer is in the engaged position. A second location (e.g., a second end) of the outlet channel may be positioned in a portion of the cleaning and/or disinfecting apparatus in order to deliver the contents from the outlet channel to another component with the cleaning and/or disinfection apparatus. For example, as will be discussed in further detail later, the outlet channel may be operatively coupled to a hose or other tubing that further transports the contents of the container to a desired location within the cleaning and/or disinfecting apparatus.

The piercer assembly may remain in the engaged position for a predetermined amount of time (e.g., at least as long as it will take to empty the contents of the container), and then the motor will change to its second orientation, causing the piercer to retract from the engaged position and return to a normal or resting position. In some embodiments, one or more content sensors are provided within the apparatus to determine when the contents of the container are present, and to determine when the contents of the container are no longer present in the container, the reservoir, and/or the outlet channel. When the content sensors determine that the contents of the container have been emptied, the content sensors may transmit a control signal configured to cause the motor to operate in its second orientation, retracting the piercer from its engaged position. Thereafter, the container may be removed and replaced by another container to allow for additional cleaning and/or disinfecting of additional probes.

Moreover, in some embodiments of the invention water, or another liquid, may be delivered into the container, through the piercer, the tube, or other like feature, to mix with the contents of the container, in order to provide the cleaning and/or disinfecting. That is, the liquid may mix with the contents of the container and the mixture of the liquid and contents of the container are supplied for cleaning and/or disinfecting as described in further detail herein.

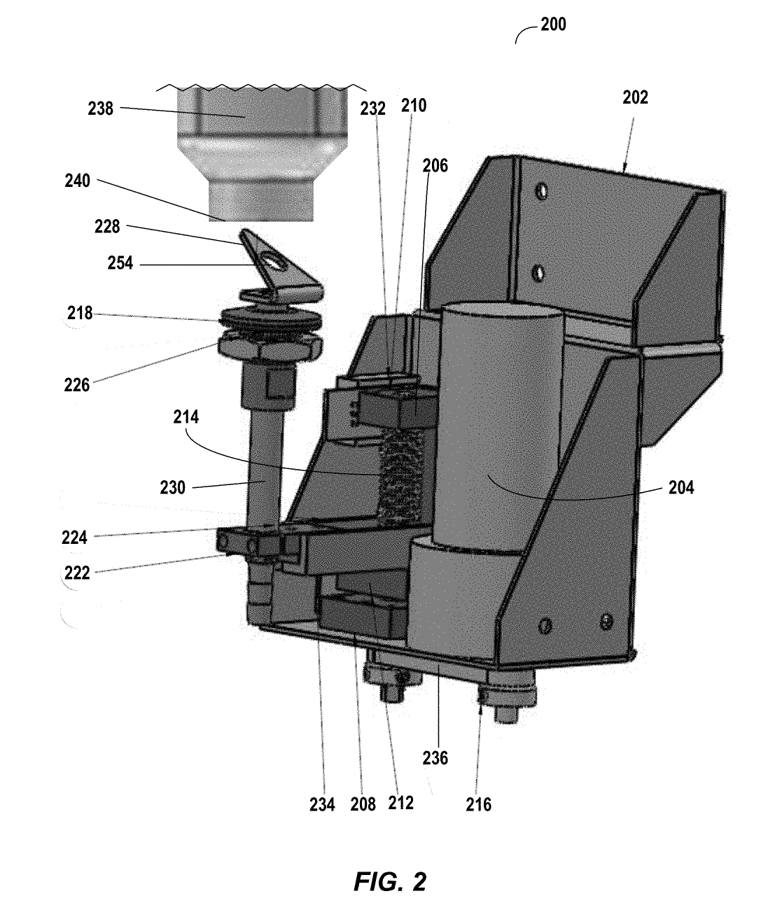

FIGS. 2-4 illustrate various views of the piercing apparatus 200 in accordance with embodiments of the present invention. The piercing apparatus 200 may generally comprise a piercing assembly and a drive assembly. The piercing apparatus 200 may further comprise a bracket 202 that may be operatively coupled to an apparatus (e.g., a cleaning and/or disinfecting apparatus), and that functions as a housing for at least a portion of the other components of the piercing apparatus 200.

The drive assembly may comprise an actuator, such as a motor, and drive mechanisms of the piercing apparatus 200. The motor 204 (or another type of actuator) may be operatively coupled to the bracket 202, and may be operatively coupled to drive mechanisms, such as at least one pulley 216. The drive mechanisms may further comprise a belt 236 that is operatively coupled to the pulley 216 and to a lead screw 214, such that the belt 236 forms a tight band as it wraps around the pulley 216 and the lead screw 214. In this way, as the motor 204 runs, it may cause the pulley 216 to rotate, and friction between the pulley 216 and the belt 236 may cause the belt 236 to travel around the pulley 216. As noted above, the motor 204 may be any type of actuator. As such, the actuator may utilize hydraulic, pneumatic, electric, thermal, magnetic, mechanical, or other like means for causing the belt to rotate the lead screw, thereby moving the piercer. Alternatively, the actuator may utilize these means to actuate the piercer in other ways, such as but not limited to moving a piston and/or linkages, or other like drive mechanisms. As the belt 236 also wraps around the lead screw 214, friction between the belt 236 and the lead screw 214 may cause the lead screw 214 to rotate in the same rotational direction as the pulley 216. In some embodiments (not shown), the belt 236 may cross itself between the pulley 216 and the lead screw 214 such that the lead screw 214 rotates in an opposite rotational direction as the pulley 216. In some embodiments, additional components such as guides (e.g., nuts, ridges, depressions, disks, etc.) for the belt 236 may be positioned on or substantially near the pulley 216 and/or the lead screw 214, such that the belt 236 remains substantially in the same place as it rotates around and/or with the pulley 216 and the lead screw 214.

At least a portion of the lead screw 214 may be housed within a carriage 206, with a carriage base 208 operatively coupled to the bracket 202. The carriage base 208 and the bracket 202 may comprise one or more apertures at least as large as the width of the lead screw 214, such that at least a portion of the lead screw 214 may extend below the bracket 202 to engage with the belt 236.

The lead screw 214 may have one or more portions with helical or spiraling ridges throughout its length. Additionally or alternatively, the lead screw 214 may have one or more portions of its length that are smooth, and do not have ridges. For example, the lead screw 214 may not have any ridges at the portion of the lead screw 214 that interacts with the belt 236, such that the belt 236 is not manipulated or moved by the ridges of the lead screw 214. Similarly, a separate, smooth portion of the lead screw 214 may be operatively coupled to a carriage end 210 at the top of the carriage 206, where the lead screw 214 is allowed to rotate within the carriage end 210. Therefore, the rotation of the lead screw 214, as caused by the movement of the belt 236, may simply cause the lead screw 214 to spin substantially along its longitudinal axis without any displacement along the longitudinal axis.

In some embodiments, the drive assembly (or the piercer assembly) also comprises a guided nut 212, with an aperture of substantially the same size as the lead screw 214, and having at least one ridge and/or depression along the same angle as the lead screw 214. The guided nut 212 may also be configured in a square, block, or other shape such that the guided nut 212 rests alongside at least one wall, side, pole, guide, track or other component of the carriage 206 that prevents the guided nut 212 from rotating along with the lead screw 214. The wall or other guiding feature of the carriage 206 may also comprise a sliding surface that allows the guided nut 212 to slide longitudinally along the side of the carriage 206, substantially along the longitudinal axis of the lead screw 214. Therefore, when the lead screw 214 is turned by the belt 236, the guided nut 212 is blocked from rotating by the side of the carriage 206, and the ridges of the lead screw 214 push the ridges and/or depressions of the guided nut 212 in a direction along the longitudinal axis of the lead screw 214.

In this way, the operation of the motor 204 can cause the guided nut 212 to travel linearly with respect to the lead screw 214. In some embodiments, a first operating state of the motor 204 ultimately causes the guided nut 212 to travel linearly along the lead screw 214 in a first direction defined as from the carriage base 208 towards the carriage end 210. In some embodiments, a second operating state of the motor 204 ultimately causes the guided nut 212 to travel linearly along the lead screw 214 in a second direction defined as from the carriage end 210 towards the carriage base 208. In some embodiments, a third operating state of the motor 204 is an idle or powered-off state, where the motor does not exert any forces upon the pulley 216.

The guided nut 212 may be operatively coupled to any piercing assembly or piercer, such that as the motor 204 causes the guided nut 212 to rise linearly along the lead screw 214, the piercing assembly or piercer may also rise linearly, where it then may engage a container to pierce the container, thereby emptying the contents of the container.

In some embodiments, and as illustrated in FIG. 2, the guided nut 212 may be operatively coupled to a clamp, through a clamp base 224 and/or a clamp cap 222. The clamp base 224 and the clamp cap 222 may be operatively coupled to one another to form a clamping device configured to be operatively coupled to the guided nut 212. The clamping device may also be configured to provide a clamping force upon a portion of the piercing assembly, such as a tube 230 (e.g., a hollow piercing member, or the like). While a clamping device comprising a clamp base 224 and a clamp cap 222 are illustrated in FIG. 2, it should be understood that any type of clamping device may be utilized to operatively couple to the guided nut 212 and securely hold a tube 230. In some embodiments, the guided nut 212 is also the clamping device, such that a tube is operatively coupled to the lead screw 214.

In some embodiments, other coupling techniques may be used to operatively couple the guided nut 212 to the tube 230. For example, the tube 230 and the guided nut 212 may be a single piece of material (e.g., a single metal component, a single plastic or other polymer material component). In other embodiments, the tube 230 and the guided nut 212 may be operatively coupled to each other through means other than clamping. For example, if the tube 230 and the guided nut 212 are metallic, then the two components could be welded together to create a larger metallic piece. Similarly, the tube 230 and the guided nut 212 may be glued together to form the securing bond. These are merely examples of different ways that the tube 230 and the guided nut 212 may be operatively coupled together, and it should be understood that any method of joining a tube with a different component can be used. As will be described in more detail below, the tube 230 may comprise an outlet channel 244 through the interior of the tube 230. In such embodiments, the coupling mechanism between the tube 230 and the guided nut 212 should not break a wall of the tube 230 such that fluid, gas, or other contents that may be passing through the outlet channel 244 will not escape through the wall of the tube 230.

While the drive mechanisms of the piercing apparatus 200 are described herein as using a motor 204, one or more pulleys 216, a lead screw 214, and a guided nut 212, it should be known that other drive mechanisms may be used to cause movement of the tube 230 and/or other components of the piercer apparatus 200. For example, the drive mechanisms may use gears, direct connection to a belt, rack and pinion drive, linear motor, linkages that move a piston, or other like drive mechanisms.

The piercing assembly of the piercing apparatus may comprise a tube 230 (or other member), a piercer, a guide, and one or more sealing members (and the other components previously described above). The tube 230 may extend above and below the clamp base 224 and the clamp cap 222. In some embodiments of the invention, the tube 230 may be solid, and as such, the contents of the container drain into the reservoir and are delivered from the reservoir to the medical device after the piercer pierces the container.

In other embodiments, the tube may be a hollow tube 230 used to deliver the contents of the container. As such, a bottom of the tube 230 (e.g., a second location or second end of the tube) may comprise ridges or cone-shaped features for operatively coupling to a hose or other tubing system. When the tube 230 is operatively coupled to a hose or other tubing system, the outlet channel 244 defined within the tube 230 may extend into (or around, or butt up to) the hose or other tubing system such that any contents of a container 238 traveling through the outlet channel 244 may continue to pass through the hose or other tubing system. In such embodiments, the hose or other tubing system may direct the contents of the container 238 to one or more other portions of an apparatus, such as a cleaning and disinfecting apparatus. In other embodiments, the bottom of the tube 230 is open, such that contents from the container 238 traveling through the outlet channel 244 may be emptied out the bottom of the tube 230. In some such embodiments, the end of the tube 230 may be positioned over one or more medical devices or otherwise deliver the contents of the container 238 to one or more medical devices, such as a probe, such that the contents of the container 238 contact the one or more medical devices.

In still other embodiments, the tube 230 may have two or more channels (e.g., two separate channels) and/or there may be multiple tubes 230 such that one channel and/or one tube acts as a liquid supply tube that delivers liquid to the container after the container is pierced. In this way the liquid (e.g., water, or the like) is delivered to the container to mix with the contents of the container and a second channel and/or tube acts as a delivery tube to deliver the mixture of the liquid and the contents of the tube to the reservoir, the medical device, or other like location. As such, in some embodiments there may also be multiple piercing apparatuses 200 and/or multiple piercing assemblies and/or drive assemblies. As such, the container may be pierced in one or more locations (e.g., two locations) in these embodiments using one or more piercing assemblies and/or one or more drive assemblies in order to supply the liquid to mix with the contents of the container and/or to allow the contents of the container (e.g., with or without mixing with the liquid to drain from the container). It should be further understood that the contents of the container have been generally described herein as a liquid, but it should be understood that in some embodiments of the invention the contents may be a powder, which when mixed with a liquid supply may form the cleaner and/or disinfectant, which is then delivered to the desired location.

Moreover, in some embodiments of the invention the container may have two chambers, that is, one for a cleaner and one for a disinfectant. As such, the different portions of the container may be pierced individually by the one or more piercing apparatuses 200 (or the one or more piercing assemblies and/or one or more drive assemblies) as needed depending on if/when the cleaner and/or disinfectant is needed for the process of cleaning and disinfecting the medical device (e.g., probes, or the like).

In some embodiments, the top of the tube 230 (e.g., a first location or first end of the tube) extends in substantially the same direction as the lead screw 214. As such, a portion of the tube 230 at and/or near the top of the tube 230 may be operatively coupled to a guide 226, a tube seal 220 (e.g., an o-ring, or the like that allows for sealing and movement of the tube with respect to the guide), a reservoir seal 218 (e.g., a gasket, or the like that seals the interface of the reservoir and the piercer apparatus), and/or a piercer 228. The guide 226 may be any nut, washer, plate, band, clasp, cylinder, block, other structure, or combination thereof that is operatively coupled to the tube 230 and is configured to direct, steer, or otherwise guide the tube 230, and components of the tube 230, to and/or through an opening or aperture in a reservoir 242 of an apparatus and/or to secure the reservoir seal 218 to prevent or reduce leakage from the reservoir. In some embodiments, one or more tube seals 220 are operatively coupled to the guide 226 and the tube 230. In some such embodiments, the inner surface of the guide 226 may be defined by a continuous groove, such that at least a portion of a tube seal 220 may rest within the groove of the inner surface of the guide 226. The tube seal 220 may be a packing, a toric joint, in a loop shape with a round cross-section. The tube seal 220 may be comprised of an elastomer such that when compressed between two or more parts (e.g., the guide and the tube), the compressed tube seal 220 may create a fluid-tight and/or air-tight seal.

The reservoir seal 218 may be comprised of any material that is generally flat, but malleable such that when compressed between two objects, it creates a fluid-tight and/or air-tight seal. As such, the reservoir seal 218 fills in any less-than-perfect mating surfaces between the two objects. In some embodiments, the reservoir seal 218 may sit on top of and/or be operatively coupled to the guide 226 and the reservoir. In certain embodiments, the guide 226, operatively coupled with the reservoir seal 218, may be configured to press up against an outer surface of a reservoir 242. As such, the guide 226, the reservoir seal 218, and/or the outer surface of the reservoir 242 may form a fluid and/or air-tight seal. In some embodiments there may be a reservoir seal above an opening in the reservoir 242 and a second reservoir seal below the opening in the reservoir 246, such that a first guide 226A may be a male guide and a second guide 226B may be a female guide (or vice versa) and the one or more sealing members 218 may be located between guide and/or both sides of the reservoir 242 to create a reservoir sealing system.

In other embodiments, a container seal of the piercer assembly may press up against a portion (e.g., a side, a lip, etc.) of a container 238, such that the container seal, and/or the portion of the container 238 forms a fluid and/or air-tight seal. In some such embodiments, the top of the guide 226, the piercer, the container seal, or other components in the piercer assembly may form a seal with the portion of the container 238, while a reservoir seal 218 (located at a lower position than the top of the guide 226) forms a seal between the guide 226 and a portion of the reservoir 242.

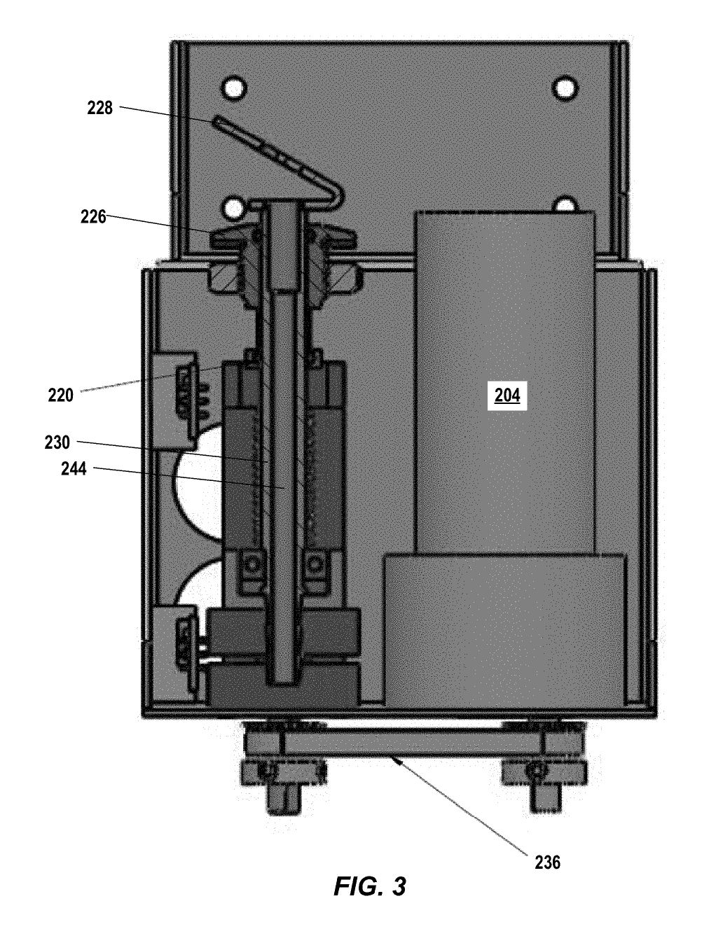

In some embodiments, a piercer 228 is operatively coupled to and extends from at least a portion of the tube 230. The piercer 228 may be any protruding strip, prong, or other sharp object (e.g., a chisel, file, knife, spike, nail, blade, or the like), that is operatively coupled at one end to at least one of the tube 230 (or the guide 226, the container seal, or other component in other embodiments of the invention). In some embodiments, and as illustrated in FIGS. 2-4, the piercer 228 may comprise a first piercer portion (e.g., a substantially flat surface) that is operatively coupled to the tube 230, where the piercer 228 also comprises a second piercer portion (e.g., an angled portion) that forms a point capable of piercing one or more objects. In some embodiments, the piercer 228 comprises one or more piercer apertures 250, as shown in FIGS. 2 and 3. In some embodiments, the piercer apertures 250 allow contents of a container 238 to pass through the piercer 228 and enter the reservoir and/or the outlet channel 244 of the tube 230. In some embodiments, the piercer 228 does not comprise any piercer apertures 250, such that the contents of a container 238 do not have a passage through the piercer 228 to the reservoir and/or outlet channel 244 of the tube 230. In some embodiments, the piercer 228 has a single piercer aperture 254 that aides in allowing contents of a container 238 to pass through a first portion of the piercer 228, but does not provide a passage to the outlet channel 244 to the tube 230.

The piercer 228 may be configured to pierce a container 238 when the tube 230 (and therefore the piercer 228) are moved under the power of the motor 204 such that at least a portion of the top of the piercer 228 is pressed against the container 238. In some embodiments, the piercer 228 is sharp enough or strong enough to pierce (e.g., pry open, pierce, split, or otherwise separate) a portion of a wall of the container 238 from itself, such that contents stored within the container 238 empty out of the container 238 at the opening created by the piercer 228. Therefore, in some embodiments, the container 238 may be substantially locked in place by one or more container locks (e.g., clasps, snaps, clamps, lids, ridges, and the like), such that the container 238 does not move upwards as the piercer 228 is pressed up against the container 238. In some embodiments, the container 238 and/or the contents of the container 238 are heavy enough that gravity holds the container 238 in place while the piercer 228 presses up against the container 238 and pierces the container 238.

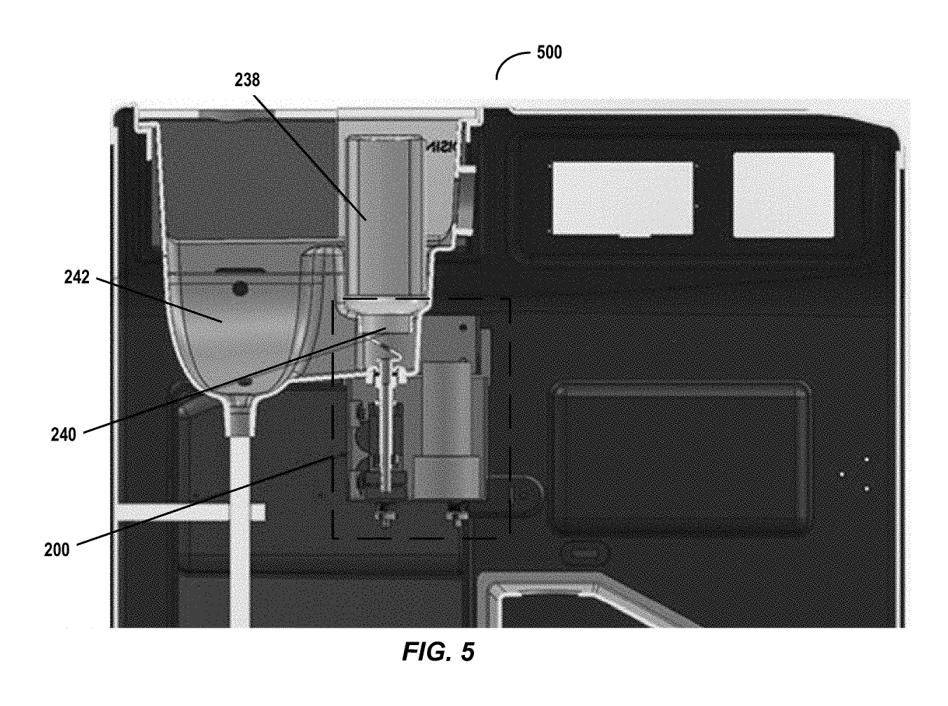

In some embodiments, the container 238 comprises at least one container cover 240, as illustrated in FIGS. 2 and 5. In some embodiments, the container cover 240 is comprised of the same material as the container 238. In other embodiments, the container cover 240 is comprised of a material that is more malleable, fragile, or otherwise easier to pierce than the material of the rest of the container 238. The container cover 240 may be a foil seal that can be easily pierced by the piercer 228, when the piercer 228 is moved up to and through the container cover 240 by the activation of the motor 204 (or other actuator).

The engaged position of the piercer apparatus 200 may comprise the piercer 228 being positioned through and within the container 238 and/or the container cover 240, while the guide 226, the reservoir seal 218, and/or the tube seal 220, form a seal between the tube 230 and the reservoir 242, and the outlet channel 244 is either shut or is not present. In such embodiments, the contents of the container 238 are emptied by gravity out of the container 238, and move or otherwise flow downward into and across the reservoir 242, where the contents of the container 238 may then interact with a medical device such as a probe.

The engaged position, in other embodiments, comprises the piercer 228 being positioned through and within the container 238 and/or the container cover 240, while the guide 226 and/or the piercer 228, and the container seal form a seal with the container 238. As described above, the outlet channel 244 may transport the contents of the container 238 to a different reservoir or basin, to a hose or other channel through which the contents of the container 238 may pass, or otherwise directs the contents of the container 238 to a desired location within an apparatus. As the reservoir 242 positioned under the container 238 may have previously been used to hold other fluids and/or gases that should not interact with the contents of the container 238, a container seal may need to be in place between the tube 230 and guide 226 structure and a portion of the reservoir 242 where the tube 230 and guide 226 pass through the reservoir 242. Therefore, in some embodiments, the guide 226 may extend from its container seal with the container 238 (e.g., the top surface of the guide 226 along with the container seal) down at least to a point where the tube 230 passes through the reservoir 242 at the guide 226 and reservoir seal 218. One or more of the reservoir seals 220 may be positioned on this extended guide 226 such that a seal is formed between the guide 226 and the reservoir 242. As such, the reservoir 242 is sealed off from the contents of the container 238, while the contents of the container 238 are sealed off and protected from dilution and/or contamination from other materials and chemicals that may be present in the reservoir 242. This separation may be important when cleaning and/or disinfecting medical devices that require a specific amount and/or concentration of cleaning product and/or disinfectant to be introduced to a medical device.

In some embodiments, the piercer apparatus 200 further comprises at least one sensor bracket 232 that houses one or more sensors 234 and/or circuitry for reading measurements taken by the one or more sensors 234, communicating the measurements taken by the one or more sensors 234 to one or more external devices (e.g., via a wireless connection such a WiFi, Bluetooth, RFID, and the like, a wired connection, or a combination of the two), and/or for controlling the motor 204.

One or more position sensors 234 may be configured to measure distances and/or directions of movement for the guided nut 212, the tube 230, the guide 226, and/or the piercer 228. The one or more position sensors 234 may be any sensor type configured to measure a distance of travel, an acceleration of travel, a direction of travel, or the like.

The one or more position sensors 234, the sensor bracket 232, and/or a monitoring system for the one or more position sensors 234 may be programmed to track the movement of one or more components of the piercer apparatus 200, and to indicate when the piercer apparatus is in a certain engaged position. In some embodiments, the engaged position of the piercer apparatus 200 is a configuration where the piercer 228 has pierced a container 238, the guide 226 and the reservoir seal, the container seal, and/or the tube seal form a fluid and/or an air-tight seal with the container and/or the reservoir. When the one or more position sensors 234 determine that the piercer apparatus 200 has reached the engaged position, the one or more position sensors 234 and/or the sensor bracket 232 may transmit one or more control signals to the motor 204 (either directly to the motor 204 or through a network connection with the motor 204) configured to cause the motor 204 to stop causing the pulley 216 to rotate. Therefore, when the one or more position sensors 234 determine that the piercer apparatus 200 is fully engaged with the container and/or the reservoir, the system may shut off the motor 204 and/or otherwise lock the piercer apparatus 200 in place. These one or more position sensors 234 may be configured to monitor, detect, or otherwise measure a location or amount of engagement of at least a portion of the piercer 228, an actuation of an actuator (e.g., the motor 204), or any other component of the piercer apparatus 200 whose location is indicative of an amount of engagement of the piercer 228 with the container 238.

In some embodiments, the piercer apparatus 200 may remain in its engaged position for a predetermined amount of time, generally at least as long as it will take to empty the contents of a pierced container 238. In some embodiments, one or more content sensors (not shown) may be operatively coupled to the piercer 228, the reservoir 242, the tube 230, and/or a compartment of a cleaning and/or disinfecting apparatus such that the content sensors sense and detect the presence of the contents of the container 238. For example, a content sensor may be a sensor configured to detect the presence of a fluid, a specific chemical, a certain concentration of a chemical, and the like. In some embodiments, the content sensors may be any wet/dry liquid sensors. The content sensors can communicate with the rest of the piercer apparatus 200 to indicate when the content of the container 238 is present, and when the content of the container 238 is no longer present. Therefore, in some embodiments, after the position sensor(s) 234 has shut down the motor 204, the content sensor(s) causes the motor 204 to operate in its second configuration, returning the piercer 228 from the container 238, and back to a resting or normal position.

In other embodiments of the piercing apparatus, the outlet channel 244 of the tube 230 or additional tubing downstream of the tube 230 may be operatively coupled to a pump (not illustrated) that may regulate the flow the contents being discharged from the container. As such, the cleaning and/or disinfecting apparatus, or user thereof, my regulate the flow of the contents of the container after the container is pierced.

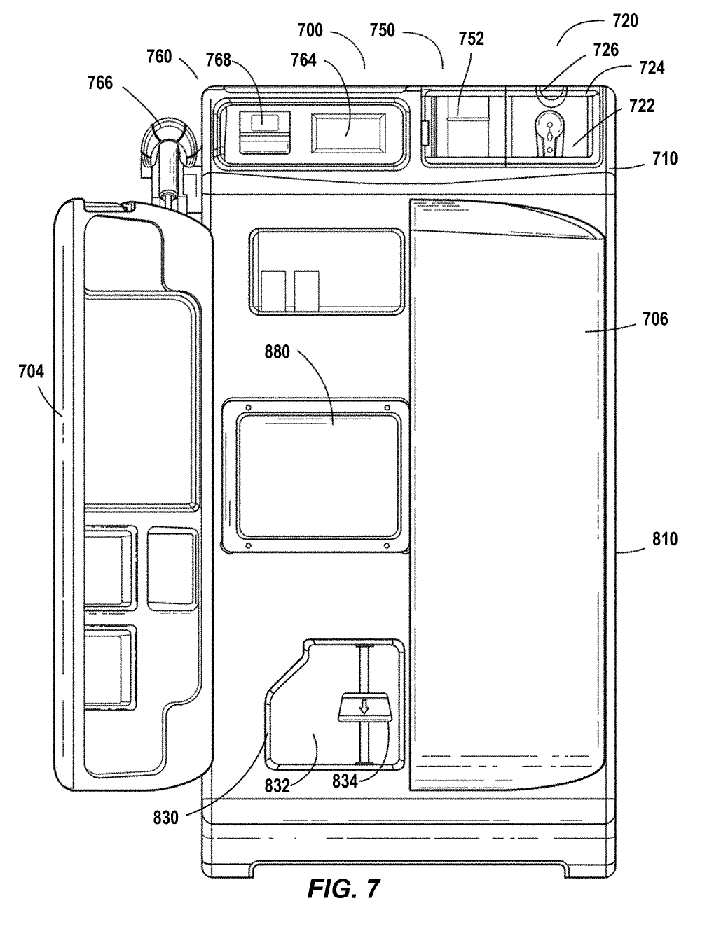

In some embodiments, the piercing apparatus 200 is a component of a larger cleaning and disinfecting apparatus 500, as illustrated in FIG. 5. One specific use of the piercing apparatus 200 is for use in piercing containers of disinfectant as part of a larger apparatus and method of cleaning and disinfecting TEE probes. However, it should be understood that the apparatuses and methods of the present invention may be utilized on other types of probes, or other types of medical devices. This one specific use of utilizing the piercing apparatus 200 to aide in cleaning and disinfecting TEE probes is described herein, and as illustrated in FIGS. 7-11. The apparatuses and methods described herein utilize a cleaner to soak and/or flush the probes to remove bioburden, soil, and the like (e.g., hemoglobin, carbohydrates, proteins, endotoxin, or the like) (described collectively herein as “foreign material”) from the probe after it is removed from a patient. The cleaner may be an enzymatic detergent that has bacteriostatic properties to inhibit bacterial growth in the apparatus 700 (e.g., within the cleaner assembly 740, supply lines, fluid circuit, or the like). The multiple enzymes in the cleaner rapidly attack soils, and have low foam properties for effective recirculation within the apparatus 700. The probe is rinsed after cleaning to remove or substantially remove the residual cleaner from the probe and the rest of the fluid circuit. After rinsing a high-level disinfectant process is applied to the probe. The high-level disinfectant can be stored in a disinfectant container, and the piercing apparatus 200 is configured to open the disinfectant container, as described above and with respect to FIGS. 7-10. The high-level disinfectant soaks and/or flushes the probe for a specified amount of time to disinfect the surface of the probe, and thereafter the probe is thoroughly rinsed to remove or substantially remove any remaining disinfectant from the probe or from within the rest of the fluid circuit. The patient never interacts with the apparatus 700, as such the apparatus 700 provides a layer of insulation between the patient and the cleaner and the disinfectant through both physical barriers as well as the air filter assembly 880, which is described in further detail later.

The term cleaner used herein may describe the cleaner in its form before it is mixed with water to form the cleaner solution, and/or the cleaner solution. It should be understood that the use of the term cleaner may be substituted with the term cleaner solution throughout this application, and as such this specification may describe that the cleaner itself and/or the cleaner solution (e.g., the cleaner mixed with water) may be utilized within the process steps or within the components of the apparatus 700 described herein. Likewise, the term disinfectant used herein may describe the disinfectant in its form before it is mixed with water to form the disinfectant solution, and/or the disinfectant solution. It should be understood that the use of the term disinfectant may be substituted with the term disinfectant solution throughout this application, and as such this specification may describe that the disinfectant itself and/or the disinfectant solution (e.g., the disinfectant mixed with water) may be utilized within the process steps or within the components of the apparatus 700 described herein. Moreover, it should be understood that the term fluid circuit described herein may include the components and tubes within the apparatus in which the cleaner, disinfectant, and/or the water passes through.

The present invention provides for the cleaning and disinfecting of a probe within the apparatus 700. The apparatus 700 comprises a housing 702 that at least partially encloses the components of the apparatus 700, which both securely hold the probe to avoid damage to the probe and also control the processes for directing the cleaner and the disinfectant through the flow paths of the apparatus 700 to clean and disinfect the probe.

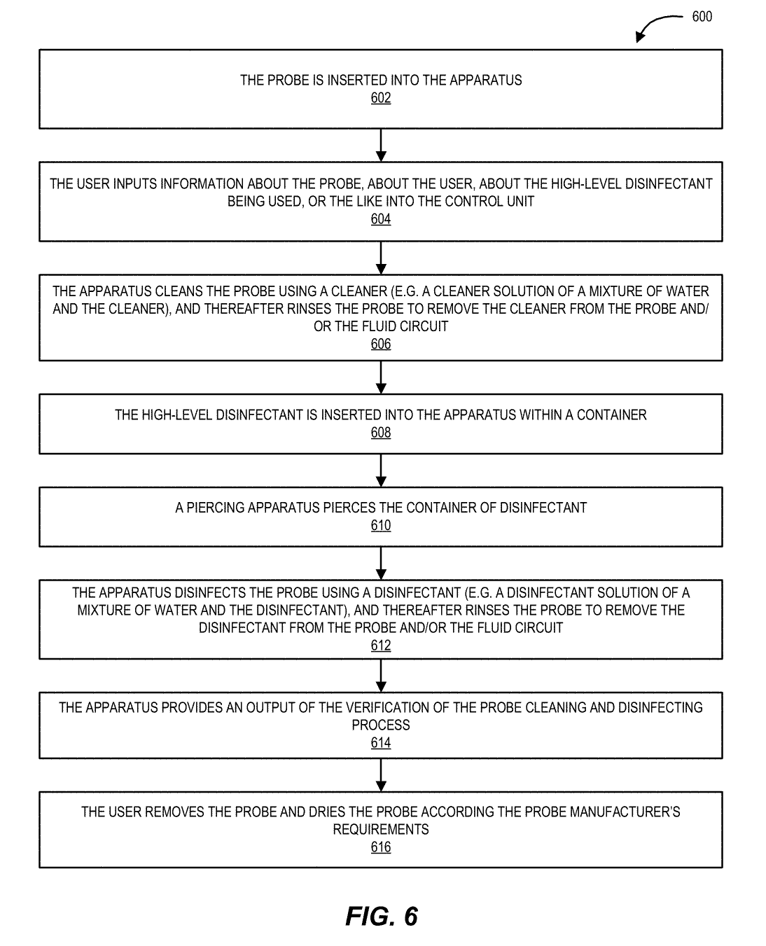

FIG. 6 illustrates a high level process flow 600 for cleaning and disinfecting a TEE probe. As illustrated by block 602 a single soiled TEE probe is inserted into the apparatus 700. As will be discussed in further detail later, the probe is secured safely within a probe reservoir assembly 720 and/or the probe tube 730. In other embodiments of the invention, the apparatus 700 may be able to accommodate multiple probes at a time, for example within one or more probe reservoir assemblies 720 and/or probe tubes 730.

As illustrated by block 604, a user inputs information into the apparatus 700 through a control unit assembly 760, which is described in further detail later. The information may be related to the probe being cleaned and disinfected, the user operating the apparatus 700, the cleaner and/or the high-level disinfectant being used to clean or disinfect the probe, the duration of time and/or temperatures for cleaning, rinsing, disinfecting, and final rinsing, or other process steps. In some embodiments, these programmed times for cleaning, rinsing, disinfecting, final rinsing, and/or other like process steps are pre-programmed into the apparatus 700. As such, in some embodiments when the processing temperatures and/or times are pre-programed, the user does not have the ability to change these inputs. If the pre-programed process is not followed or the cycle is interrupted before completion, the cycle may be aborted and a failure notice may be provided to the user (e.g., failure ticket is printed, displayed on an interface, or the like). After the inputs are set, the user may begin the cleaning, rising, disinfecting, and final rinsing steps of the process.

Block 606 of FIG. 6 illustrates that the apparatus 700 cleans the probe by applying a pre-determined amount of cleaner to the probe from the cleaner assembly 740, as will be discussed in further detail later. The cleaner may be supplied as a dose from a multi-use supply (e.g., 50 or more or less doses within a container, or other like dose amount), or as a dose from a single use supply (e.g., single use container). In some embodiments of the invention the single use container and/or the dosage from a multi-use container may be utilized without the need to add additional water (e.g., a ready to use dose), while in other embodiments the single use container and/or the dosage from a multi-use container (e.g., a concentrated dose) may be mixed with water to form a cleaner solution, in order to disinfect the probe. In some embodiments the cleaner is mixed with filtered water from the water filter assembly 830 to create the cleaner solution before being used to clean the probe. Moreover, in some embodiments the cleaner is heated to approximately 40 degrees C. In other embodiments the cleaner is heated to a temperature in the range of 35 degrees C. to 45 degrees C., inclusive. However, it should be understood that the temperature to which the cleaner is heated may be within this range, overlap this range, or fall outside of this range in alternate embodiments of the invention. As explained in further detail later the cleaner may be heated before it is mixed with the water, after it is mixed with the water, or the water may be heated before it is mixed with the cleaner.

The cleaner (e.g., the heated cleaner solution) is delivered to the probe, and thereafter recirculated back through the fluid circuit to the probe again in one or more cleaner cycles to clean the probe before the probe is disinfected. As explained in further detail below a heater assembly 860 may continuously heat the cleaner, as it is recycled through the fluid circuit in order to maintain the temperature of the cleaner at the desired temperature range. In some embodiments of the invention, the cleaner may remain stagnant for a period of time to allow the probe to soak within the cleaner. In some embodiments of the invention, the cleaner may be applied to the probe (e.g., delivered and recirculated) for a minimum of five (5) minutes to remove the foreign material from the surface of the probe. In other embodiments, the cleaner may be applied to the probe for less than or greater than five (5) minutes. Each use of the cleaner may be a single use, and thus, the cleaner waste is discarded to the one or more drains 824 after a cleaning cycle. In one embodiment the cleaner may have a dedicated cleaner drain to keep the cleaner waste from mixing with the disinfecting waste, which is described in further detail later. Moreover, in some embodiments a lint filter may be used to remove the foreign material from the cleaner (e.g., cleaner solution) during each cycle of the cleaner through the fluid circuit or after the cleaning step is complete. In some embodiments, the cleaner may be delivered to the probe and discharged from the apparatus in one or more cycles (e.g., a single cycle), and thereafter, a new second cleaner (e.g., the heated cleaner solution) may be delivered to the probe in a second cycle, and so on (e.g. third cleaner delivered in a third cycle, or the like).

After cleaning, the probe, as well as the components and tubes of the fluid circuit, are thoroughly rinsed by water from the water filter assembly 830 and/or the water inlet 822, in one or more cleaner rinsing cycles. In some embodiments, after each cleaner rinsing cycle or after the cleaner rinsing step, the cleaner rinsing waste is also discarded to the one or more drains 824, such as the dedicated cleaner drain. The water used to rinse the probe may also be heated in some embodiments of the invention (e.g., to the same or similar temperatures as described with respect to the cleaner and/or the disinfectant described below). During the cleaner rinsing step the water may be recycled through the fluid circuit, or new water may be used within each cycle of the cleaner rinsing step.

As illustrated by block 608 of FIG. 6, a predetermined amount of high-level disinfectant is loaded into the apparatus 700 within a container. For example, in some embodiments of the invention the high-level disinfectant is a single use container (e.g., bottle, package, or the like) that is utilized once and the container is disposed of after the disinfecting step is completed.

As illustrated in block 610 of FIG. 6, a piercing apparatus component of the apparatus 700 pierces the container of disinfectant. For example, the piercing apparatus described in block 610 is the same piercing apparatus 200 described in FIGS. 1-5. The single use container may be punctured inside of the apparatus 700 to contain and minimize splashes, spills, and vapors within the apparatus 700. In other embodiments of the invention, the disinfectant may be delivered from a disinfectant supply that has more than a single use, and that is already loaded into the apparatus 700. In some embodiments of the invention the single use container and/or the dosage from a multi-use container may be utilized without the need to add additional water (e.g., a ready to use dose), while in other embodiments the single use container and/or the dosage from a multi-use container (e.g., a concentrated dose) may be mixed with water to form a disinfectant solution that is used to disinfect the probe.

In some embodiments of the invention, the piercing apparatus component is repositioned through a manual process. For example, a user may operate the piercing apparatus component using a lever, a handle, a line, a button, etc. to move the piercing component towards the container and to pierce a portion of the container.

Similarly, in some embodiments of the invention, the piercing apparatus component is stationary within the apparatus and the container is configured within the apparatus to be moved into, onto, or to otherwise engage with the piercing apparatus, which allows the container to be pierced by the piercing apparatus. In such embodiments, the container may be inserted into the apparatus, into an installed position. A safety (e.g., a lock that allows the container to move, and the like) may be released to permit the container to be moved into a piercing position from the installed position (e.g., manually or automatically), where the container is pierced when in the piercing position.

Alternatively, in some embodiments, the container may be at least partially external to the apparatus (e.g., the container may begin fully outside of the apparatus), and can be inserted manually into a container receptor of the apparatus (e.g., an opening, slide, channel, and the like) and further moved toward a stationary piercing apparatus until the container is moved into the piercing position, thereby piercing the container to begin the cleaning and/or disinfection process. In some such embodiments, the container receptor of the apparatus and the container form an air-tight and/or fluid-tight seal when the container is engaged with the container receptor and/or piercing apparatus. In this way, the seal can keep the contents of the container (e.g., gas and/or fluid contents that splatter or are otherwise expelled from the container) from escaping the apparatus.

Block 612 of FIG. 6 illustrates that the apparatus 700 heats the high-level disinfectant (e.g., the disinfectant solution) to approximately 38-40 degrees C., inclusive, before being delivered to the probe for disinfecting. In some embodiments the disinfectant may be heated utilizing the heater assembly 860. The disinfectant may be heated before being combined with the water, after being combined with the water, or the water may be heated before being combined with the disinfectant. In some embodiments the temperature may be within, outside, or overlapping the recited temperature range of the disinfectant (e.g., disinfectant solution).

In some embodiments of the invention, the disinfectant (e.g., disinfectant solution) may be delivered to the probe and recirculated back through the system to the probe in one or more disinfectant cycles to disinfect the probe. In some embodiments of the invention, the disinfectant may remain stagnant for a period of time to allow the probe to soak within the disinfectant. The disinfectant may be heated each time it is recirculated through the fluid circuit in order to maintain the temperature of the disinfectant to the desired temperature range. In some embodiments, the disinfectant may comprise Glutaraldehyde, and make up 2.65% of the disinfectant solution, with the remainder comprising of water and/or other components. In other embodiments of the invention a different type of disinfectant may be utilized and/or the amount of the disinfectant element may be below or above the recited 2.65%. The disinfectant may be applied (e.g., delivered and recycled) to the probe for at least five (5) minutes at the desired temperature range. In other embodiments of the invention the minimum amount of soaking and/or flushing time may be below or above the at least five (5) minutes. In one embodiment, after disinfecting the probe the disinfectant (e.g., disinfectant solution) may have a dedicated disinfectant drain in order to keep the disinfectant waste from mixing with the cleaner waste, which is described in further detail later. In some embodiments, the disinfectant may be delivered to the probe and discharged from the apparatus in a one or more cycles (e.g., a single cycle), and thereafter, a new second disinfectant (e.g., the heated disinfectant solution) may be delivered to the probe in a second cycle, and so on (e.g. third disinfectant delivered in a third cycle, or the like). The disinfectant waste may be required to be kept separate from the cleaner waste (and other rinsing water waste) because the disinfectant waste may have to be chemically inactivated, depending on the requirements of different facilities or areas of use.

Thereafter, the water (e.g., heated water) thoroughly rinses the disinfectant off the probe, as well as out of the components and tubes of the fluid circuit, in one or more disinfectant rinsing cycles. In some embodiments, after each disinfectant rinsing cycle or after the disinfectant rinsing step, the disinfectant rinsing waste is also discarded to the one or more drains 824, such as the dedicated disinfectant drain. The water used to rinse the probe may also be heated in some embodiments of the invention (e.g., to the same or similar temperatures as described with respect to the disinfectant and/or cleaner). During the disinfectant rinsing step the water may be recycled through the fluid circuit, or new water may be used within each cycle of the disinfectant rinsing step.

As was the case with the cleaner step, in some embodiments a lint filter may be used to remove the foreign material from the disinfectant (e.g., disinfectant solution) or rinsing water during each cycle of the disinfectant or rinsing water, or after the disinfectant step or rinsing steps are complete.

The water used herein for creating a cleaner solution, a disinfectant solution, or for rinsing may be 0.2-micron filtered bacteria free water. In other embodiments of the invention the water used to create the cleaner solution, the disinfectant solution, or for rinsing the probe may be water that is less than or greater than the 0.2 micron filtered bacteria free water.

In some embodiments of the present invention, since the disinfectant used with each cycle is received from a single use disinfectant container, no monitoring of the disinfectant's potency is required, nor is there any requirement for daily testing of the disinfectant. The single use containers are created with the desired potency, and as such no measurement of the disinfectant solution is needed before it is utilized for disinfection.

Block 614 of FIG. 6 illustrates that the apparatus 700 may provide output of the verification of the cleaning and disinfecting of the probe. The verification of the cleaning and disinfecting of the probe may be based on the use of sensors that measure temperature (e.g., temperature sensors 790), wet/dry areas of the apparatus 700 (e.g., wet/dry sensors 890), or other like process or apparatus parameters, which are described in further detail later. For example, the apparatus 700 may provide the results of diagnostic tests that confirm that the probe has been properly disinfected because the probe was cleaned, rinsed, disinfected, and rinsed again with solutions for the desired durations and at the desired temperatures. The output verification may occur in the form of a printed document. However, in other embodiments of the invention, the output may be displayed on a screen that is operatively coupled to the apparatus 700 (e.g., within the apparatus 700 or on a computer display operatively coupled to the apparatus 700). In other embodiments of the invention, the output may be e-mailed, texted, instant messaged, or transferred through any other electronic means in order to provide the output to the desired user.

After the rinsing process in block 612, and after, before, or during the output verification process of block 614, the probe is removed from the apparatus and dried according to the probe manufacturer's instructions, as illustrated by block 616 in FIG. 6. After drying, the apparatus 700 is ready for a new cycle immediately after the preceding cycle is completed. A single cycle (e.g., from insertion of a probe to the removal of the probe after cleaning and disinfecting) may take approximately 25 minutes. It should be understood that in other embodiments of the invention the cycle may be less than 25 minutes or greater than 25 minutes.

FIGS. 7-10 illustrate various views of the apparatus 700 in accordance with one embodiment of the present invention. The apparatus 700 may comprise a head assembly 710 and a base assembly 810, as illustrated in FIGS. 7-9. The base assembly 810 holds and provides support for the internal components of the apparatus 700, while the head assembly 710 houses and supports the probe (e.g., when inserted into the apparatus 700), the disinfectant, the cleaner, and some of the components required to complete the cleaning and disinfecting processes. It should be understood that in some embodiments, the apparatus 700 may be configured in a single housing assembly that is a combination of the head assembly 710 and the base assembly 810, and will work in the same or similar way as is described herein. Moreover, it should also be understood that the head assembly 710 and the base assembly 810 may be split into two or more additional assemblies and work in the same or similar way as is described herein. It should also be understood that the assemblies and individual components thereof, which are described as being located in the head assembly 710 may in fact be located in the base assembly 810 and vice versa, and moreover, these assemblies and components thereof will still operate in the same or similar manner as described herein.

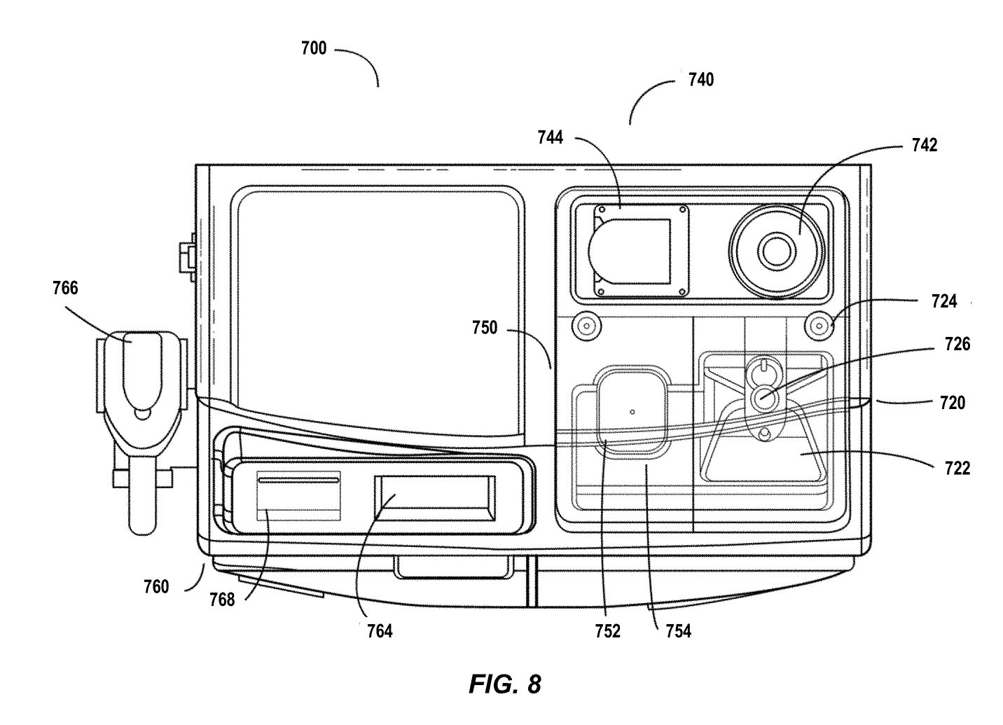

In one embodiment, the head assembly 710 comprises a probe reservoir assembly 720, as illustrated in FIGS. 7 and 8. The probe reservoir assembly 720 may comprise a probe reservoir 722 and a probe reservoir cover 724. The probe reservoir cover 724 comprises a probe cover aperture 726 for receiving a probe with a first portion (e.g., the portion that is or may be inserted into the body of a patient) and a second portion (e.g., the portion that includes the handle, electrical connection, and/or cord of the probe). The probe reservoir 722 may be operatively coupled to a probe tube 730 (e.g., a j-tube, or the like) that is operatively coupled to the water inlet and drain assembly 820 located in the base assembly 810. In some embodiments of the invention, the probe tube 730 may be a part of the probe reservoir assembly 720. The probe tube secures the first portion of the probe by housing the probe within the probe tube 730 to prevent the probe from being damaged during the cleaning, rinsing, disinfecting, and final rinsing processes. As such, the first portion of the probe may be completely secured within the probe tube 730; however, in some embodiments at least a portion of the first portion of the probe may be secured within the probe reservoir 722 of the probe reservoir assembly 720. The second portion of the probe (e.g., the handle, electrical connection, and/or cord of the probe) may be secured within the probe reservoir 722 and/or probe reservoir cover 724. In some embodiments of the invention only the first portion of the probe is cleaned and disinfected in the probe reservoir assembly 720 (e.g., within the probe reservoir 722) and/or within the probe tube 730 coupled to the probe reservoir assembly 720. The second portion of the probe (e.g., the handle, chord, electrical connection, or the like) may sit outside of the probe reservoir assembly 720 (e.g., probe reservoir 722) and may be cleaned and disinfected by the user before or after the cleaning and disinfecting process is applied to the first portion of the probe. The second portion of the probe may be damaged if it is submerged in the solutions and water that are used within the apparatus 700 for cleaning, disinfecting, and rinsing the first portion of the probe.

The first portion of the probe (e.g., within the probe reservoir 722 and/or within the probe tube 730) is thoroughly cleaned, rinsed, disinfected, and finally rinsed in order to allow the probe to be reused on subsequent patients. It should be understood that in other embodiments of the invention the probe reservoir assembly 720 may be combined with other assemblies, split into one or more multiple assemblies, or configured in other ways in order to provide a means for securing the probe, and in particular the first portion of the probe, for cleaning and disinfecting, while preventing the second portion of the probe from being subjected to potentially damaging conditions (e.g., being submerged in cleaning solutions, disinfecting solutions, or water). In other embodiments there may be multiple probe reservoirs assemblies 720, or multiple probes may be fit within the probe reservoir assembly 720, in order to clean multiple probes at once.

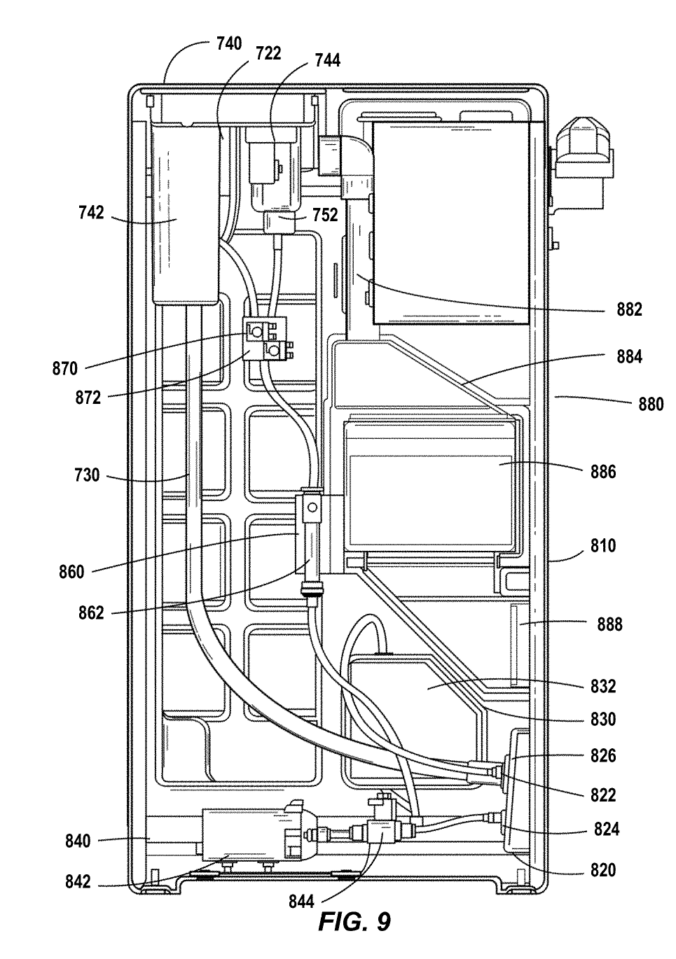

The head assembly 710 may further comprise a cleaner assembly 740, as illustrated in FIGS. 8 and 9. The cleaner assembly 740 comprises a cleaner reservoir 742 and a cleaner dispenser 744 (e.g., a cleaner pump, or other like means for distributing the cleaner solution). The cleaner assembly 740 is operatively coupled to the fluid circuit. For example, the cleaner assembly 740 may deliver the cleaner directly or indirectly to the tubing of the fluid circuit, to the probe reservoir assembly 720, to the probe tube 730, to the water filter assembly 830, to the main pump assembly 840, and/or to the heater assembly 860. As previously discussed the cleaner may be mixed with the water in the fluid circuit, heated, delivered to the probe, and recycled through the system one or more times. The cleaner (e.g., cleaner solution) is used to remove the foreign material from the probe before it is disinfected using the disinfectant. The cleaner removes material from the surface of the probe to allow the disinfectant in the process step described below to disinfect the surface of the probe that may contain residual foreign material that cannot be seen by the naked eye.

The head assembly 710 may further comprise a disinfectant assembly 750, as illustrated in FIGS. 7-9. The disinfectant assembly 750 may comprise a disinfectant reservoir 752, a disinfectant cover 754, and a means for puncturing (e.g., a piercing apparatus like piercing apparatus 200 comprising a piercer 228, a projection, punch, blade, scissor, or the like) a disinfectant container containing the disinfectant. The disinfectant reservoir 752 is configured for receiving the disinfectant container (e.g., a one-time use bottle or package), which provides the disinfectant for mixing with the water, and for delivery to the probe assembly 720 (e.g., probe reservoir 722) for disinfecting the probe. In some embodiments, the piercing apparatus 200 pierces the disinfectant such that the disinfectant solution travels through the outlet channel 244 of the tube 230, and travels to the disinfectant reservoir 752. In this way, the disinfectant reservoir 752 does not need to be next to be close to other reservoirs and/or the probe(s), such that the disinfectant is not contaminated and does not contaminate other areas of the apparatus 700. The disinfectant (e.g., disinfectant solution) is utilized to remove, kill, or otherwise sterilize the external surfaces of the probe that may have residual foreign material (e.g., residual bioburden, soil, and other like biological material) that is left on the surface of the probe after the initial cleaning process.

It should be understood that in other embodiments of the invention the cleaner assembly 740 and the disinfectant assembly 750, may be embodied in a single assembly, combined with other assemblies, split into two or more multiple assemblies, or configured in other ways in order to clean and disinfect the probe.

As illustrated in FIG. 7, the head assembly 710 may further comprise a control unit assembly 760 for housing the electronic components of the apparatus 700. The control unit assembly 760 may comprise a user interface 762 that is utilized to control the apparatus 700 and the electrical components for running the user interface 762. In some embodiments the user interface 762 may comprise a touchscreen 764 and/or keypad, a scanner 766 (e.g., barcode scanner, QR code scanner, digital image scanner, or other like scanner), and an output device 768 (e.g., printer, output interface, connection to computer system, touchscreen 764, or the like). The touchscreen 764 and/or the keypad enable the user to initiate operation of the unit. The scanner 766 allows the user to input data to the apparatus 700, for example data related to the cleaner, disinfectant, probe, user, process durations and/or temperatures, or the like, as previously discussed with respect to FIG. 1. The output device 768, such as the printer, provides the user with a diagnostic output of the machine condition, operating status, success or failure of the cleaning and disinfecting of the probe, and/or the process parameters (e.g., temperatures, wet/dry conditions, durations of the steps of the process, or the like) of the cleaning and disinfecting process. For example, the output may include the temperatures from the temperature sensors 790, wet/dry indications from the wet/dry sensors 890, a filter replacement notification, duration of the cleaning, rinsing, disinfecting, and/or final rinsing process steps, or the like. It should be understood that in other embodiments of the present invention the control unit assembly 760 may be combined with other assemblies, separated into two or more other assemblies, or configured in other ways in order to allow the user to control the apparatus 700 and receive output regarding the cleaning and disinfecting of the probe.

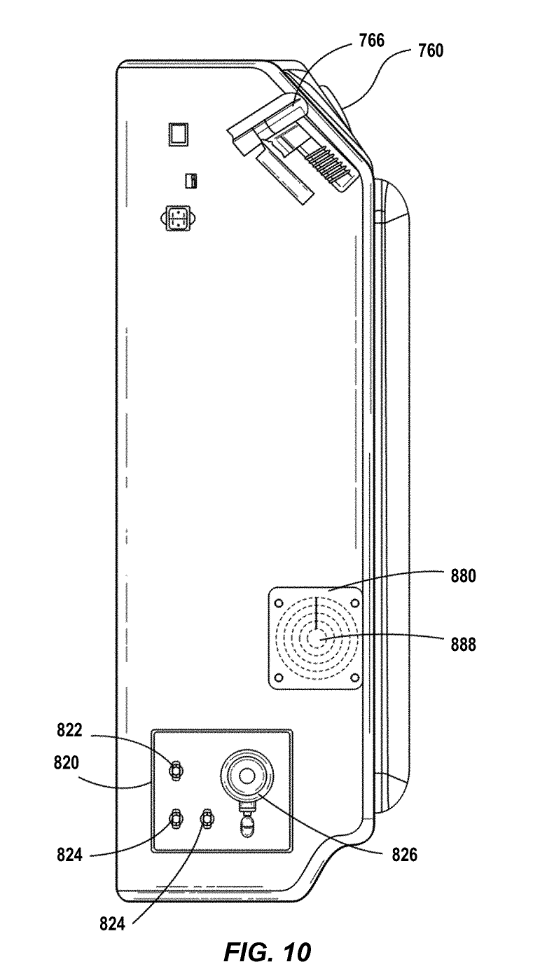

The body assembly 810 may comprise a water inlet and drain assembly 820, as illustrated in FIGS. 9 and 10. The water inlet and drain assembly 820 may comprise a water inlet 822, one or more drains 824, and a lint trap 826, which may all be operatively coupled to the housing 702 of the base assembly 810. The water inlet 822 provides a location to receive water and provide the water to the water filter assembly 830, which is discussed in further detail below. The drains 824 allow for the removal of used cleaner waste, disinfectant waste, and/or rinse water waste (e.g., cleaner rinsing waste or disinfecting rinsing waste) that are flushed through the system, and specifically through the probe tube 730 that operatively couples the probe reservoir 722 and the water inlet and drain assembly 820. The one or more drains 824 may include a specific drain for disinfectant waste and disinfecting rinsing waste, and a specific drain for cleaner waste and cleaner rinsing waste. The lint trap 826 is used to catch and separate the foreign material (e.g., bioburden, soil, and other biological material) from and the cleaner, disinfectant, and/or water (e.g., cleaner waste, disinfectant waste, cleaner rinsing waste, disinfecting rinsing waste, or the like) received from the probe reservoir 722 and the probe tube 730 as the probe is cleaned, rinsed, disinfected, and final rinsed. For example, the lint trap 826 may be used to remove the foreign material from the cleaner, disinfectant, and/or water as they are recycled through the fluid circuit or as they are drained out of the system after completion of the cleaning, rinsing (e.g., cleaner rinsing), disinfecting, or final rinsing (e.g., disinfecting rinsing) steps. The lint trap may be emptied as necessary, and the used cleaner waste, disinfectant waste, and/or water waste may be disposed of or recycled as needed. It should be understood that in other embodiments of the present invention the water inlet and drain assembly 820 may be combined with other assemblies, separated into two or more other assemblies, or configured in other ways in other embodiments of the invention in order to provide the source of water to the apparatus 700 and to remove the waste products of cleaning, rinsing, disinfecting, and final rinsing from the apparatus 700. In one embodiment the water inlet and drain assembly 820 (or another assembly) may be operatively coupled to (e.g., contain, be connected to, or the like) a water supply tank that houses at least a portion of the water for use as the water supply for mixing with the cleaner or disinfectant, and/or used as the rinse water. In still other embodiments, one or more waste tanks may be operatively coupled to the drain assembly 820 (or another assembly). The one or more waste tanks may be utilized to hold the waste from the cleaner and/or cleaner rinse cycles, and/or the disinfectant and/or disinfectant rinse cycles. The waste may be held within the one or more waste tanks while the waste material is inactivated to protect the public waste system. This embodiment may be particularly useful for the disinfectant and/or disinfectant rinse water, which may be required to be inactivated before disposing of the waste.

The body assembly 810 further comprises a water filter assembly 830, as illustrated in FIG. 9. The water filter assembly 830 may comprise a water filter housing 832, a water filter 834, and water tubes and valves that receive water from the water inlet and drain assembly 820 and delivers the water directly or indirectly to the probe reservoir assembly 720, or other assemblies as discussed herein. The water filter 834, as previously discussed may be a 0.2 micron water filter that filters water from particles that are greater than 0.2 microns. In other embodiments of the invention the water filter may be configured to filter particles that are less than or greater than 0.2 microns. In some embodiments, when the water filter 834 requires replacement, the apparatus 700 may undergo pasteurization at temperatures of approximately 70-75 Celsius for approximately thirty (30) minutes in order to flush out the apparatus 700 and ensure that the water filter 834 is operating properly before the apparatus is used for further cleaning and disinfecting. In other embodiments of the invention the temperature of pasteurization may be inside, outside, or overlapping this temperature range and/or the duration may be above or below thirty (30) minutes of pasteurization. It should be understood that in other embodiments of the present invention the water filter assembly 830 may be combined with other assemblies, separated into two or more other assemblies, or configured in other ways in order to supply the water for mixing with the cleaner to create the cleaner solution, for mixing with the disinfectant to create the disinfectant solution, or for rinsing the probe and/or the fluid circuit before and after cleaning and/or disinfecting.

As illustrated in FIG. 9, the body assembly 810 may further comprise a main pump assembly 840 that is used to pump water from the water filter assembly 830 and/or the water inlet 822 to the probe reservoir assembly 720 as needed to clean, disinfect, and rinse the probe and/or the fluid circuit. The main pump assembly 840 may also be utilized to pump at least some of the cleaner or disinfectant to the probe reservoir assembly 720 along with the water. The main pump assembly 840 may comprise a pump 842 and a pump manifold 844. The pump manifold 844 is utilized to operatively couple the pump 842 to the water filter assembly 830, as well as directly or indirectly to the water inlet and drain assembly 820, the heater assembly 860, the disinfectant valve assembly 870, the probe reservoir assembly 720, the probe tube 730, the cleaner assembly 740, and/or the disinfectant assembly 750. It should be understood that in other embodiments of the present invention, the main pump assembly 840 may be combined with other assemblies, separated into one or more other assemblies, or configured in other ways in order to pump the water, cleaner, or disinfectant needed for cleaning, disinfecting, and rinsing, to the probe reservoir assembly 720.

The body assembly 810 may further comprise a heater assembly 860, as illustrated in FIG. 9. The heater assembly 860 may comprises a heater 862 and tubes and valves for heating and supplying the heated water and heated solutions described herein to the probe reservoir assembly 720, the probe tube 730, and to recycle the water and solution described herein throughout the fluid circuit and back to the probe reservoir assembly 720 and/or the probe tube 730. The heater assembly 860 may be operatively coupled to the probe reservoir assembly 720, the probe tube 730, the cleaner assembly 740, the disinfectant assembly 750, the water filter assembly 830, and/or other assemblies described herein. In some embodiment, the heater assembly 860 may be located between the disinfectant valve assembly 870 (discussed below) and the main pump assembly 840.

The heater 862, in some embodiments may heat the mixture of the water and the cleaner (e.g., cleaner solution), or the mixture of the water and the disinfectant (e.g., disinfectant solution) after the solutions are created. In other embodiments the heater 862 may heat the water before the water is mixed with the cleaner or disinfectant, or when the water is applied to the probe reservoir assembly 720 or probe tube 730 by itself for rinsing. In still other embodiments of the invention the heater 862 may heat the cleaner and/or the disinfectant before they are mixed with the water. The water and/or the solutions are heated to the desired temperatures discussed herein by the heater 862 before being delivered to the probe assembly 720 or probe tube 730 to clean, disinfect, or rinse the probe. It should be understood that in other embodiments of the present invention, the heater assembly 860 may be combined with other assemblies, separated into one or more other assemblies, or configured in other ways in order to heat the water and/or the cleaner and disinfectant, for cleaning, disinfecting, and rinsing the probe in the probe reservoir assembly 720 or the probe tube 730.

A disinfectant valve assembly 870 may also be included in the body assembly 810, as illustrated in FIG. 9. The disinfectant valve assembly 870 may comprise a disinfectant valve 872 and supply tubes that receive the disinfectant from the disinfectant assembly 740 and the water from the main pump assembly 840, and deliver the mixture of water and disinfectant (e.g., the disinfectant solution) to the probe reservoir assembly 720. It should be understood that in other embodiments of the present invention the disinfectant valve assembly 870 may be combined with other assemblies, separated into two or more other assemblies, or configured in other ways in order to mix the water and disinfectant together and to supply the disinfectant solution to the probe reservoir assembly 720 and/or the probe tube 730.

The body assembly 810 may further comprise an air filter assembly 880, as illustrated in FIGS. 9 and 10. The air filter assembly 880 may comprise an air filter duct 882, an air filter housing 884, an air filter 886, and a fan 888. The fan 888 may pull any fumes from the cleaner, and more particularly from the disinfectant, away from the user located near the apparatus 700, and especially near the insertion point of the probe in the probe reservoir assembly 720. As such, the air filter duct 882 may be operatively coupled to the probe assembly reservoir 720, the probe tube 730, the cleaner assembly 740, the disinfectant assembly 750, and/or generally within the housing of the apparatus 700. The air filter duct 882 may be operatively coupled to the fan 888, for example through the air filter housing 884 and/or the air filter 886, in order to draw the fumes into the air filter housing 882 through the air filter 886 to remove or reduce potentially dangerous components from the fumes, and to exhaust the air out of the apparatus 700 through the fan 882. In one embodiment the air filter 886 is a carbon air filter that absorbs the glutaraldehyde in the fumes of the disinfectant. However, in other embodiments of the invention the air filter 886 may be any type of filter that removes or reduces potentially dangerous components from the fumes of the cleaner and/or the disinfectant. It should be understood that in other embodiments of the present invention, the air filter assembly 880 may be combined with other assemblies, separated into one or more other assemblies, or configured in other ways in order to remove fumes from the apparatus 700. It should be understood that the air filter 886 may comprise an RFID tag, or other indication illustrating the type of filter that may be used to remove the fumes form the cleaner and/or disinfectant. In other embodiments of the invention detector sensors may be utilized to indicate when the filter 886 has reached, or is about to reach, its end of useful life. The detector sensors may be operatively coupled to the control unit assembly 760, and thus, the user may be notified when the filter 886 has reached, or is about to reach, its end of useful life.

The housing 702 of the apparatus may comprise various features to access the components of the apparatus. In one embodiment the housing 702 may include a housing door 704 that may be utilized to access the air filter assembly 880 for accessing and replacing the air filter 886, and to access the water filter assembly 830 for accessing and replacing the water filter 834. In another embodiment the housing 702 may include a housing service panel 706 that allows a user to access the one or more assemblies contained within the housing 702.

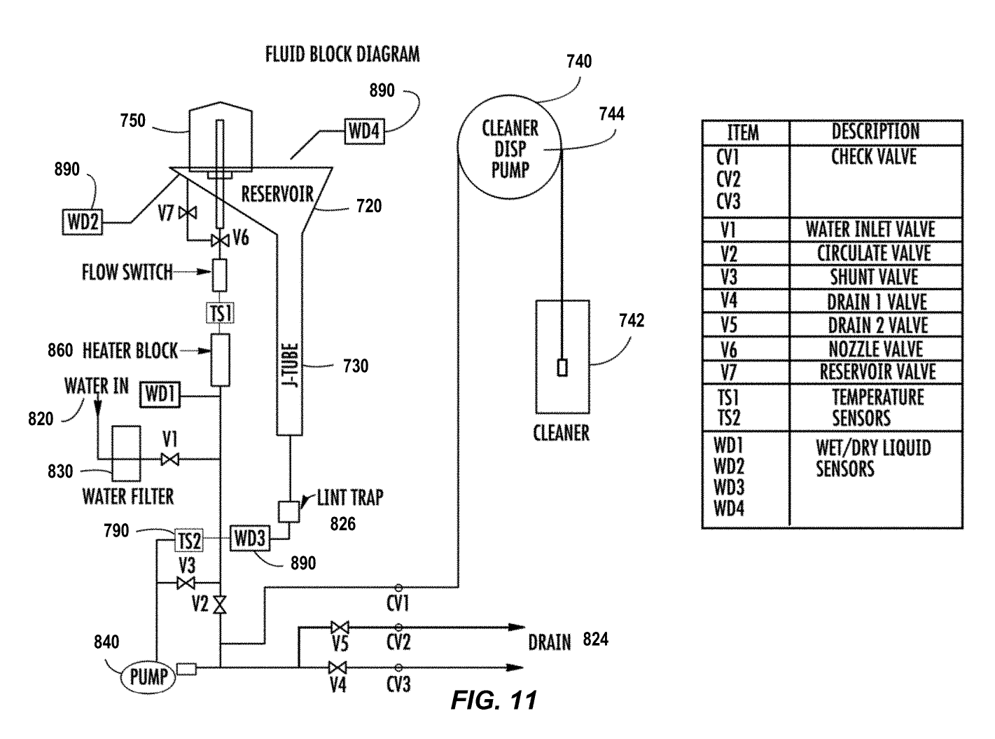

As generally discussed with respect the various assemblies described herein, each assembly that transfers water, cleaner, disinfectant, a mixture of these fluids, or the like may utilize supply and return lines (e.g., tubing) to operatively couple the assemblies together and to transfer the fluids from one assembly to another. As previously discussed the assemblies and the supply and return lines may be described herein as the fluid circuit. The supply and return lines may be as illustrated in part in FIG. 9, and further illustrated in one embodiment of the invention, in the schematic diagram of FIG. 11, which is discussed in further detail below.

As illustrated by FIG. 11, the probe is inserted in the probe reservoir assembly 720 and the probe tube 730, and the cleaner is dispersed (e.g., through the cleaner pump 744) into the supply lines for the eventual delivery of the cleaner to the probe reservoir assembly 720. The cleaner mixes with the water (e.g. from the water inlet and drain assembly 820 or the water filter assembly 830) to form the cleaner solution. The main pump assembly 840 pumps the cleaner solution to the heater assembly 860 in order to heat the cleaning solution to the desired temperature, and thereafter the main pump assembly 840 pumps the heated cleaning solution into the probe reservoir assembly 720. The cleaning solution fills the probe reservoir 722 and/or the probe tube 730 to clean the first section of the probe (e.g., portion inserted into the patient). In some embodiments, as illustrated in FIG. 11 the main pump assembly 840 recycles the cleaning solution from the probe reservoir 722 and/or the probe tube 730 back to the heater 860 to keep the cleaning solution at the desired temperature range, and eventually back to the probe reservoir 722 and/or the probe tube 730 for further cleaning in a continuous loop for the desired amount of cleaning time. The cleaning solution clears the foreign material (e.g., bioburden, soil, and other like biological material) from the surface of the probe by soaking and/or flushing the cleaning solution through the probe reservoir assembly 720 and/or the probe tube 730 in one or more cycles. The lint trap 826 may filter the foreign material out of the cleaning solution during each of the one or more cycles or after the cleaning step is completed. After cleaning the recycling of the cleaner solution is shut off, and the cleaner solution drained out of the apparatus through the one or more drains 824 (e.g., the dedicated cleaner drain). Thereafter, the main pump assembly 840 may then pump water (e.g., heated water) from the water inlet and drain assembly 820 and/or the water filter assembly 830 into the probe reservoir assembly 720 and/or the probe tube 730 to thoroughly rinse the cleaner solution off of the probe. The cleaning rinse water waste may also be drained out of the apparatus through the one or more drains 824 (e.g., the dedicated cleaner drain).

The user may then insert an unopened single use disinfectant container into the disinfectant assembly 750. The single use disinfectant container may be inserted at the beginning of the process, or in some embodiments may be inserted after the cleaning process is completed. Inserting the disinfectant container after the cleaning process may prevent the disinfectant from mixing with cleaning solution in the probe reservoir assembly 720 during the cleaning operation. In some embodiments of the invention the disinfectant may be TD-5, which is used by other systems for disinfecting medical instruments. The dosage of the disinfectant within the container may be pre-determined in order to remove the need to test the disinfectant in a reservoir before each disinfecting step in order to identify if the disinfectant has the desired potency. However, in other embodiments of the invention the container of disinfectant may be a multi-use bottle that the disinfectant assembly 750 may regulate in order to apply the desired amount to the probe assembly 720. In some embodiments, the disinfectant container is pierced by a piercing apparatus like piercing apparatus 200, such that the disinfectant solution is emptied from the disinfectant container and delivered to the disinfectant reservoir 752.

As illustrated in FIG. 11 the water is heated first and then mixed with the disinfectant to create the disinfectant solution before being distributed to the probe reservoir assembly 720 and/or the probe tube 730. The main pump assembly 840 recycles the disinfectant solution from the probe reservoir assembly 720 and/or the probe tube 730 to the heater assembly 860 to maintain the desired temperature range of the disinfectant solution before being delivered back to the probe reservoir assembly 720 and/or the probe tube 730. The disinfectant solution soaks and/or flushes the probe and the fluid circuit for at least five (5) minutes. In other embodiments the disinfectant is mixed with water before the heater assembly 840 heats the disinfectant solution, and before the disinfectant is first delivered to the probe reservoir assembly 720 and/or the probe tube 730.

After the disinfecting step, the probe is then thoroughly rinsed using the water (e.g., heated or unheated) for 10-12 minutes, to remove the disinfectant solution from the probe. In other embodiments of the invention the probe may be rinsed within a duration range that is inside of this range, outside of this range, or overlaps this range. A previously discussed the probe is removed from the apparatus 700 and dried according to the manufacture's specifications.

FIG. 11 also illustrates a number of valves at various locations that are utilized to open and close the fluid circuit to deliver or cut off the supply of water, cleaner, disinfectant, cleaner solution, disinfectant solution, cleaner solution waste, disinfectant waste, cleaner rinse water waste, and/or disinfectant rinse water waste to the various components of the apparatus, as described herein and as illustrated in FIG. 11. For example, in one embodiment of the invention the V1 valve is utilized for turning on/off the water supply to the system; the V2 valve is used to allow the fluid (e.g., cleaner, disinfectant, rinse water, or the like) from circulating continuously through the fluid circuit; the V3 valve may be used for permitting the fluid under pressure exiting the probe tube 730 an easier avenue of escape; the V4 valve may be used to drain the cleaner and/or cleaner rinse water; the V5 valve may be used to drain the disinfectant and/or the disinfectant rinse water; the V6 valve may be used to release the disinfectant into the fluid circuit; and the V7 valve may be utilized to release the cleaner, disinfectant, and/or rinse water into the reservoir 720.

Moreover, as illustrated by FIG. 11, check valves may be utilized to control the flow of the fluid in the fluid circuit; for example, the CV1 check valve may prevent fluid in the fluid circuit from entering the cleaner pump and/or cleaner reservoir 742. The CV2 and CV2 check valves may be used to prevent waste exiting the drain lines from returning back to the fluid circuit.

Software monitors the cleaning and disinfecting process using temperature sensors 790 and wet/dry sensors 890. As illustrated by the schematic diagram in FIG. 11, temperature sensors 790 may be located in, or at the exit of, the heater assembly 860 and after the probe tube 730 (e.g., directly after the probe tube 730, directly after the lint trap 826, or the like). In other embodiments of the invention temperature sensors 790 may be located in other areas of the apparatus 700, or more temperature sensors may be utilized in other areas. The temperature sensors 790 may provide information that allows the software to determine if the water, cleaner, and/or disinfectant is heated to the desired temperatures for the cleaning, rinsing, disinfecting, and final rinsing processes. The wet/dry sensors 890 may be located at the inlet to the heating assembly 860, at the inlet to the probe reservoir assembly 720, in the probe reservoir 722, or at the exit of the probe tube 730 (e.g., directly after the probe tube 730, directly after the lint trap 826, or the like). In other embodiments of the invention the wet/dry sensors 890 may be located in other areas of the apparatus 700. The wet/dry sensors 890 may provide information that allows the software to determine if the liquid (e.g., water, cleaner, or disinfectant) is or is not located in the desired areas during different times of the cleaning and disinfecting process. As such, the software may provide information regarding the temperatures, and the locations and duration that the fluids are present within the apparatus 700 to the user through the output device 768 (e.g., printed, displayed in an interface, or the like). Moreover, the software may indicate whether or not the probe passed the cleaning, rinsing, disinfecting, and final rinsing process steps without encountering any errors.

It should be understood that “operatively coupled,” when used herein, means that the components may be formed integrally with each other, or may be formed separately and coupled together. Furthermore, “operatively coupled” means that the components may be formed directly to each other, or to each other with one or more components located between the components that are operatively coupled together. Furthermore, “operatively coupled” may mean that the components are detachable from each other, or that they are permanently coupled together.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the just described embodiments can be configured without departing from the scope and spirit of the invention. Therefore, it is to be understood that, within the scope of the appended claims, the invention may be practiced other than as specifically described herein.

What is claimed is:

1. An apparatus for piercing a container, the apparatus comprising:
an actuator;
a drive mechanism operatively coupled to the actuator;
a piercer assembly operatively coupled to the drive mechanism, wherein the piercer assembly comprises at least a piercer; and
wherein the piercer is configured to pierce the container, thereby causing contents of the container to exit the container.

2. The apparatus of claim 1, wherein a first operational configuration of the actuator directs the drive mechanism to move the piercer towards the container.

3. The apparatus of claim 2, further comprising:
one or more position sensors configured to determine when the piercer is in the engaged position by measuring a position of the piercer or by measuring an amount of actuation of the actuator.

4. The apparatus of claim 1, wherein when the piercer is in an engaged position with the container at least a portion of the piercer has pierced the container and is located within the container.

5. The apparatus of claim 1, wherein the piercer assembly further comprises a guide and a reservoir seal, and wherein the guide and the reservoir seal are operatively coupled a reservoir associated with the container and seal the piercer assembly with respect to the reservoir.

6. The apparatus of claim 1, wherein the piercer assembly further comprises a piercer member operatively coupled to the piercer, and wherein the piercer member comprises a tube and an outlet channel within the tube configured to receive contents from the container when the piercer is in the engaged position.

7. A method for piercing a container, the method comprising:
moving a piercing assembly, wherein the piercing assembly is moved toward the container, and the piercing assembly comprises at least a piercer;
determining that the piercer is in an engaged position, wherein the engaged position occurs when the piercer has pierced a portion of the container;
providing contents of the container that exit the container to a medical device; and
retracting the piercer from the engaged position to a disengaged position.

8. The method of claim 7, wherein moving the piercing assembly further comprises:
manually moving the piercing assembly toward the container.

9. The method of claim 7, wherein moving the piercing assembly further comprises:
moving the piercing assembly through a drive assembly, wherein the drive assembly comprises an actuator operatively coupled to a drive mechanism, and wherein the piercer is operatively coupled to the drive mechanism.

10. The method of claim 9, wherein the piercing assembly further comprises a guide and a reservoir seal, and the reservoir seal and the guide seal a portion of the piercing assembly within the reservoir; wherein providing the contents of the container to the medical device comprises allowing the contents to flow into the reservoir associated with the container; and wherein the reservoir is configured to collect the contents of the container and direct the contents of the container to the medical device.

11. The method of claim 9, wherein the piercing assembly further comprises a piercer member operatively coupled to the piercer, a guide, a container seal, and an outlet channel in the piercing member; wherein providing the contents of the container to the medical device comprises allowing the contents to flow into the outlet channel of the piercer member; and
wherein the outlet channel is configured to receive the contents of the container directly from the container and direct the contents of the container to the medial device.

12. An apparatus for cleaning and disinfecting a probe, the apparatus comprising:
a probe reservoir assembly, wherein the probe reservoir assembly is configured for securing a probe for the cleaning and the disinfecting;
a cleaner assembly operatively coupled to the probe reservoir assembly, wherein the cleaner assembly is configured for providing a cleaner to the probe reservoir assembly for the cleaning of the probe before the disinfecting; and
a disinfectant assembly operatively coupled to the probe reservoir assembly, wherein the disinfectant assembly comprises a piercing apparatus comprising a piercing assembly, wherein the piercing assembly is configured for piercing a container of a disinfectant and providing the disinfectant for the disinfecting of the probe after the cleaning.

13. The apparatus of claim 12, wherein the piercing assembly is configured for manual movement.

14. The apparatus of claim 12, wherein the piercing apparatus further comprises a drive assembly configured for automatically moving the piercing assembly for piercing the container.

15. The apparatus of claim 14, wherein
the drive assembly comprises:
an actuator;
a drive mechanism operatively coupled to the actuator;
the piercer assembly comprises:
a piercer member operatively coupled to the drive mechanism; and
a piercer operatively coupled to the member; and
wherein the piercer is configured to pierce the container to allow contents of the container exit the container.

16. The apparatus of claim 15, wherein a first operational configuration of the actuator directs the drive mechanism to move the piercer towards the container.

17. The apparatus of claim 15, further comprising:
one or more position sensors configured to measure a position of the member or the piercer, or an amount of actuation of the actuator.

18. The apparatus of claim 15, wherein the piercer assembly further comprises a guide and a reservoir seal, and wherein the guide and the reservoir seal are operatively coupled to the probe reservoir assembly and seal a portion of the piercer assembly with respect to the probe reservoir assembly.

19. The apparatus of claim 18, wherein the guide comprises a male fitting and a female fitting and is operatively coupled to the member to allow the member to slide within the guide.

20. The apparatus of claim 15, wherein the piercer member is a tube and further comprises an outlet channel within the tube configured to receive contents from the container when the piercer is in the engaged position.
